(19) United States
(12) Patent Application Publication
(10) Pub. No.: US 2023/0019107 A1
(43) Pub. Date: Jan. 19, 2023

(54) REVERSIBLY INHIBITED BINDING MOLECULES

(71) Applicant: PRECISION IMMUNOTHERAPEUTICS LIMITED, Sevenoaks Kent (GB)

(72) Inventors: Colin H. SELF, Sevenoaks, Kent (GB); William Gwyn COLE, Bishops Stortford Hertfordshire (GB)

(21) Appl. No.: 17/784,767

(22) PCT Filed: Dec. 11, 2020
(86) PCT No.: PCT/GB2020/053183

§ 371 Date: Jun. 13, 2022

(30) Foreign Application Priority Data
Dec. 11, 2019 (GB) 1918230.2

(51) Int. Cl. C07K 16/28; A61P 35/00

(52) U.S. Cl. CPC C07K 16/2809; C07K 16/2803; A61P 35/00; A61K 2039/585

(57) ABSTRACT

Reversibly inhibited antibodies are disclosed, suitable for treatment of diseases such as cancer. The reversibly inhibited antibodies are such that they regain their activity on illumination, for example with UV light. Compositions containing such antibodies, methods of preparation of such antibodies and methods of use of such antibodies are also disclosed. The moiety employed to provide reversible inhibition may comprise a hydrophilic polymer such as polyethylene glycol.

### INTRODUCTION TO THE INVENTION

The present invention relates to methods of treating diseases and in particular cancers, by enhancing the effectiveness of the immune system by use of a reversibly inhibited binding molecule; to reversibly inhibited binding molecules such as antibodies or aptamers which bind to epitopes on diseased cells such as cancer cells or on cells of the immune system, and which may be used to treat diseases such as cancer by enhancing the effectiveness of the immune system, to pharmaceutical compositions containing such reversibly inhibited antibodies or aptamers which can be used for such treatments and to processes for the preparation of such antibodies and aptamers and such pharmaceutical compositions. More particularly the present invention relates to the preceding methods, materials and processes wherein treatment of cancer is effected by enhancement of T-cell activation by use of a reversibly inhibited antibody or aptamer and/or to wherein treatment of cancer is caused by directly or indirectly inducing phagocytosis of cells of a cancer by use of a reversibly inhibited antibody or aptamer following illumination of said reversibly inhibited antibody or aptamer.

### BACKGROUND TO THE INVENTION

It is known that an anti-CD3 antibody may be reversibly inhibited by attachment thereto of moieties which can be released from the reversibly inhibited antibody by treatment with light thereby restoring the anti-CD3 effectiveness of the antibody. This is described in citations 1 to 7 herein after. This permitted use of a reversibly inhibited antibody to be used to treat mice in a xenotransplant method by illuminating the antibody with light to restore its activity. Such known reversibly inhibited antibodies have not been progressed to use in a human. The skilled person noting that these known reversibly inhibited antibodies contained many exposed nitrophenyl groups covalently bound to the antibody, and being aware that attachment of nitrophenyl groups to an antibody is a known method of increasing its antigenicity, would have concerns that the disclosed reversibly inhibited antibodies would not be suitable for progression into use in the human where repeated administration would be anticipated. The skilled person would have noted that repeated use of known reversibly inhibited antibodies in the same animal was not disclosed.

The art is replete with reports of antibodies which cause T-cell activation for killing cancer cells. Unfortunately use of such antibodies can lead to systemic toxicity issues if T-cells are caused to attack bodily tissues. A method used to try to reduce such side effects is to direct also the T-cell activating antibody against a binding site present on the surface of the cancer cell which is being targeted. This may be done by including a moiety which binds to a receptor or epitope overexpressed on the cancer cell. It is known that such antibodies can be labelled with folate or the like when the cancer cell expresses folate receptors or the antibody can have a second antibody function directed to an epitope expressed on the cancer cell so that it is bi-specific.

A commonly used class of antibodies proposed for T-cell activation are anti-CD3 antibodies. These antibodies often have a second functionality as directed to a receptor on a cancer cell in an attempt to reduce toxicity caused by unwanted action of the antibody. Such antibodies are described in references below.

Unless apparent from the context when the terms antibody or antibodies are used herein the term extends to binding fragments thereof which produce an anti-cancer cell effects.

However such anti-CD3 antibodies tend to have residual toxicity which may be caused by binding to cells other than the target cells or to diffusion of cytokines into other tissues.

### ANTIBODIES

A further class of antibodies that enhance the body's immune response against cancer are anti-PD-1 antibodies. These also are often suggested for use as bifunctional antibodies where the second function is directed to a receptor on the cancer cell in an attempt to reduce toxicity caused by unwanted action of the antibody. Such antibodies are described in references below.

A method by which cancers and in particular solid cancers evade destruction by the immune system is to overexpress CD47 on their surfaces. This CD47 is able to interact with SIRPα on the surface of phagocytes which produces a so called “Don't eat me” signal.

It is well established that inhibition of this CD47-SIRPα binding by an anti-CD47 antibody can lead to phagocytosis of the cancer cells (see references below).

In addition it is well established that ligation of CD47 by an anti-CD47 antibody can independently induce apoptosis of cancer cells (see references below). This also leads to increased phagocytosis since as cells become apoptotic expression of CD47 generally becomes reduced or less organised and the cells are no longer resistant to phagocytosis.

The two mechanisms set out above are not mutually exclusive and anti-CD47 antibodies may cause both apoptosis (and hence indirectly phagocytosis) as well as directly produce phagocytosis. It is also possible for anti-CD47 antibodies to cause neither effect because of binding remotely on CD47 to the epitopes resulting in apoptosis and phagocytosis (which epitopes are in close proximity or overlap). These antibodies can however serve to bind bi-specific antibodies to target cells.

Anti-CD47 antibodies can also have a second functionality directed to a receptor on a cancer cell in an attempt to reduce toxicity caused by unwanted action of the antibody.

Antibodies to cell surface nucleolin are also known to have anti-cancer effects (see references below).

Many antibodies developed against CD47, CD3 and PD-1 show promise but would nevertheless benefit from modification which could lead to their effect being directed more effectively against the cancer to be treated and/or the effect being prolonged.

As may be seen from the publications referred to herein, the preparation of antibodies (including active fragments and bi- and multispecific forms) may now be regarded as within the common general knowledge of the skilled person. Indeed a number of commercial groups offer to prepare such antibodies to the client's specifications.

Less common is the use of antibodies which target either the innate or adaptive immune system that are suitable for use together. Antibodies for use in such pairings tend to suffer from disadvantages such as difficulties in half-lives, side effects and the like.

One of the more favourable pairings is described in Ingram et al., PNAS, Sep. 19, 2017, vol 114, 10184-10189 where an anti-CD47 nanobody termed A4 was used to try to energise the effect of an anti-PD-1 antibody on malignant melanoma. Some success was achieved but the problem of antigen sink and systemic toxicity were not overcome.

Aptamers are similarly known to possess anti-cancer activity but suffer from short half-lives (see references below).

Aptly the invention provides a reversibly inhibited antibody which comprises an antibody or aptamer which stimulates the adaptive immune system to attack a cancer to which antibody is reversibly covalently bound one or preferably a plurality of light cleavable moieties which comprise a hydrophilic polymer.

Aptly the invention provides a reversibly inhibited antibody which comprises an antibody or aptamer which stimulates the innate immune system to attack a cancer to which antibody is bound one or preferably a plurality of light cleavable moieties which comprise a hydrophilic polymer.

The above antibodies may be similarly employed to treat disease of microbiological (including bacterial, fungal, viral and protozoal) origin by attacking targeted cells or viruses.

### INCORPORATION BY REFERENCE

Each citation or prior document referred to herein is incorporated herein by reference.

### INTRODUCTION TO THE INVENTION

The present invention addresses the problem of treating disease and in particular cancer for example by encouraging the body's immune system to attack the disease cells such as the cancer cells with a reduced tendency to cause attacks on other tissues of the body that might otherwise occur. Aptly, this produces a prolongation in the effective presence of the antibody. This may be provided by killing of cancer cells, for example by causing an anti-cancer cell directed enhancement of either or both of the innate immune system or the adaptive immune system.

In order to achieve this the invention provides compounds, compositions, methods, and uses for giving rise to the cytotoxic effects primarily within the locus of the cancer without giving rise to similar levels of cytotoxic effects in healthy tissues.

This is achieved by employing binding molecules (such as antibodies or aptamers) in which their anticancer effect is reversibly inhibited until illuminated, for example, in the locus of the cancer or in the blood supply leading to the cancer. Such reversibly inhibited antibodies and aptamers can have a lower tendency to give rise to counter antibodies than some prior art reversibly inhibited compounds for use in treating cancer.

The present invention also seeks to alleviate concerns about the generation of counter antibodies by at least partially masking the light cleavable moieties from the immune system unlike the prior art compounds which could cause concern regarding counter antibodies prior to light reactivation.

Although the prior art reversibly inhibited antibodies were shown to be effective anticancer agents post light induced reactivation in a mouse xenograft model, they failed to be progressed to use in the human. The potential immunity of the modified sites on the prior art antibody had the potential to be antigenic determinants which in turn could lead to removal of the reversibly inhibited antibodies by the immune system or possibly cause adverse effects on repeated exposure to the antibody.

The present invention relates to reversibly inhibited light reactivatable antibodies or aptamers in which one or more light cleavable moieties are covalently bound to an antibody which enhances the effectiveness of the immune system in killing cancer cells wherein the covalently bound light cleavable moiety comprises a hydrophilic polymer.

Also, the present invention relates to reversibly inhibited light reactivatable anti-CD47 antibodies or aptamers in which one or more light-cleavable moieties are covalently bound on anti-CD47 antibody or aptamer.

The presence of the hydrophilic polymer reduces exposure of that part of the light cleavable moiety most closely attached to the antibody or aptamer to the immune system.

The reversibly inhibited antibodies or aptamers may be administered systemically (for example by intravenous administration) or locally (for example by intratumoral or peritumoral administration).

It is presently considered favoured to employ antibodies which are reversibly inhibited by covalently binding thereto one or more generally more than one moiety which comprises a hydrophilic polymer and which is removable from the antibody by exposure to light, most aptly by exposure to UV light.

The reactivated antibodies or aptamers may enhance the innate immune system, the adaptive immune system or both as described hereinafter.

The light cleavable moieties may be as described hereinafter.

The term “antibody” used herein also includes binding fragments thereof (unless excluded by the context) and includes antibodies (and fragments) which also contain a second binding moiety, for example a bi-specific or tri-specific antibody (once again unless excluded by the context).

It is an advantage of the invention that entirely new antibodies need not be employed as the reversibly inhibited antibody may be a known antibody or aptamer, although a new antibody or aptamer may be employed is wished. It is therefore very simple to put the invention described into practice once this disclosure is read by the skilled person. The skilled person may obtain such known antibodies or aptamers, or make new antibodies or aptamers, using known methods, or by buying or commissioning antibodies or aptamers from the many laboratories and suppliers who provide antibodies and aptamers to the research community.

### SUMMARY OF THE INVENTION

The present invention provides a reversibly inhibited binding molecule such as an antibody or aptamer which comprises a binding molecule such as an antibody or aptamer which causes stimulation of the immune system to attack a cancer to which antibody is bound one or more photolabile moieties comprising a hydrophilic polymer which when subject to light are released from the binding molecule such as an antibody or aptamer which thereby regains its ability to activate the immune system.

Favourably the present invention provides a reversibly inhibited antibody binding fragment which stimulates the immune system to attack a cancer to which is reversibly covalently bound one or more light cleavable moieties comprising a hydrophilic polymer.

The reversibly inhibited antibody or aptamer may incorporate an antibody or aptamer which causes activation of the innate immune system and/or the reversibly inhibited antibody or aptamer may be derived from an antibody or aptamer which causes activation of the adaptive immune system.

Antibodies which cause activation of the innate immune system may be (a) those which cause direct activation of the innate immune system by causing direct phagocytosis of cancer cells (for example antagonistic anti-CD47 antibody which inhibits CD47-SIRPα binding) (for example by blocking “don't eat me” signals on the cancer cell) or (b) one which causes indirect activation of the innate immune system by inducing apoptosis of the cancer cells which then become subject to phagocytosis (for example an agonistic anti-CD47 antibody, an anti-nucleolin antibody or other cytotoxic antibody which induces apoptosis when bound to a cancer cell).

Antibodies which cause activation of the adaptive immune system may be those which bound to T-cells and induce them to proliferate and/or release cytotoxic cytokines. Such antibodies include those which bind to CD3, PD-1, PD-1-L and the like.

The reversibly inhibited antibodies which cause activation of the adaptive immune system can be particularly effective when employed together with an agent which induces phagocytosis of the cancer cells either directly or indirectly as the phagocytes can then present epitopes from the cancer cells to the T-cells.

The phagocytosis inducing agent may be a chemotherapeutic agent or an antibody and in particular an anti-CD47 antibody such as an antagonistic or agonistic anti-CD47 antibody and in particular a reversibly inhibited antagonistic anti-CD47 antibody (which directly induces phagocytosis of the cancer cell).

The present invention also provides a method for treating a solid cancer which comprises the administration of a first antibody and a second antibody wherein
(i) the first antibody induces phagocytosis of the cancer cells, and
(ii) the second antibody is a photoreactivatable antibody stimulator of the immune system
and illuminating the cancer whereby the second antibody becomes active and enhances killing of the cancer by the immune system.

The second antibody may be an anti-CD3 antibody.

The second antibody may be an anti-PD-1 antibody.

The present invention also provides the foregoing method wherein the first antibody is also photoreactivatable and becomes active when the cancer is illuminated.

However in cases where the first antibody is deemed of sufficiently low systemic toxicity and sufficient half-life to permit use without being rendered photoreactivatable it may be employed without being reversibly inhibited.

The present invention also provides an anti-CD47 antibody which is capable of inducing phagocytosis of the cancer cells (whether directly via inhibiting CD47-SIRPα interaction or by causing apoptosis) which is reversibly inhibited. The preferred method of reversible inhibition is by binding thereto one or more photolabile moieties which upon illumination are cleaved thereby regenerating antibody activity. Such photolabile moieties are aptly hydrophilic and favourably comprise a hydrophilic polymer.

The present invention also provides photoreactivatable first and/or second antibodies wherein the photolabile moiety comprises one or preferably more than one hypoimmunogenic photolabile moieties. Aptly the non-immunogenic photolabile moiety is hydrophilic. Favourable the non-immunogenic photolabile moiety comprises a hydrophilic polymer such as polyethylene glycol.

Generally when an antagonistic anti-CD47 antibody is employed it will comprise the whole immunoglobulin including the Fc region when the desired effect is inhibition of the CD47-SIRPα binding to produce phagocytosis. However if the desired effect is apoptosis (leading indirectly to phagocytosis) then the whole immunoglobulin or active binding fragments can be employed as the Fc effector function is not always required for this activity. If fragments are employed they will most suitably be reversibly photoreactivatable.

If desired the second antibody is an anti-CD3 or PD-1 antibody also comprising a moiety which binds to the cancer cells. Although the presence of this additional binding moiety appears conventional and mandatory in the generality of the prior art, it is not mandatory in the present invention as photoreactivation of the anti-CD3 antibody in the locus of the cancer enables this labelling to be avoided if desired. However it is often preferred to employ a CD3 or PD-1 antibody which also has a functionality to bind it to a cancer cell.

In order to maintain wide applicability of a given labelled anti-CD3 or PD-1 antibody, in one aspect of the invention the anti-CD3 or PD-1 antibody also comprises an anti-CD47 binding function such as an antibody or binding fragment thereof. In a favoured embodiment of this aspect the anti-CD47 antibody or fragment retains its phagocytosis inducing effectiveness (either direct or via induced apoptosis) so that the antibody may be regarded as a bispecific antibody causing both desired effects.

Labelled anti-CD3 or PD-1 antibodies for use with or without the first antibody are preferably reversibly inhibited, for example with a hydrophilic photolabile moiety which most aptly comprise a hydrophilic polymer such as polyethylene glycol (PEG).

The photolabile moiety may be any which leads to reactivation of the antibody on illumination with the appropriate wavelength. Most commonly this wavelength is ultraviolet, for example in the near ultraviolet which is less harmful to tissue than shorter wavelengths.

The art is replete with photolabile moieties which may be used but at present it is preferred to employ a nitro phenyl derivative which leads to the moiety be cleaved from the antibody on illumination by UV light.

The reversibly inhibited antibody or aptamers for use in this invention may be administered at the locus of the cancer or, often more conveniently, into the systemic circulation, for example by intravenous administration, for example by infusion or injection. It is helpful that the reversibly inhibited antibody or aptamer antibodies can be water soluble or solulizable. In particular an antibody or aptamer which employs PEGylated cleavable moieties tend to have sufficiently high solubility for easy administration as an intravenously administrable solution or injectable solution. Such intravenously administrable pharmaceutical compositions are normally presented in sterile form.

Cancers near a body surface, for example the skin or membrane, may be directly illuminated. Such cancers include those of the skin, oral cavity, eye and the like.

Many cancers are illuminatable by use of a fibre optic device optionally via a catheter such that the light need not be generated at the immediate locus of the cancer. Such more easily reached cancers include cancers of the testes, prostate, bladder, kidney, rectum, cervix, colon, duodenum, stomach, pancreas, liver, oesophagus, lung and breast. In some instances the fibre optic light conducting device can be positioned by simple positioning although in certain instances positioning of the device can be done using real time imaging, for example ultrasound, x-ray or the like. Some cancers may require the fibre optic to be inserted via an incision or minimally invasive surgical techniques for example in the case of ovarian or liver cancers. Illumination of the cancer may also occur during surgery, for example when removing or debulking a tumor. These methods of illumination may be used in the treatment of cancers within the body cavity such as pancreatic, ovarian, liver, bone and skull.

The cancer to be illuminated will commonly be the primary tumour but if metastases are more readily illuminatable a secondary (metastatic) tumour may be illuminated when the antibody employed is one which stimulates the adaptive immune system for example an anti-CD3 antibody. This is because of the activation of the adaptive immune system cancers at other locations as well as that illuminated will benefit from stimulation of the immune system.

Reactivation within the circulation may be employed, for example by illuminating of veins, for example at the wrist in certain circumstances.

The skilled user may administer the first antibody or administer and illuminate the reversibly inhibited first antibody (such as a CD47 antibody) or aptamer before administration and illumination of the reversibly inhibited anti-CD3 antibody in order for phagocytes to have ingested cancer cells and presented antigens for recognition by T-cells.

The present invention provides antibodies and aptamers for use in treating cancer which are reversibly inhibited and which on photoactivation regain the ability to induce phagocytosis and/or cytotoxic T-cell activity which can lead to death of cancer cells as indicated herein before.

Favourably the reversibly inhibited antibody stimulator of the adaptive immune system is a reversibly inhibited anti-CD3 and/or anti-PD-1 antibody which on photoactivation regains the ability to induce T-cell activity which can lead to death of cancer cells.

When such a reversibly inhibited antibody is used together with a phagocytosis inducing antibody the photoactivation may lead to the cleavage from the antibody of any convenient photolabile group.

A preferred photolabile group for use in antibodies which stimulate the adaptive immune systems (whether used together with a phagocytosis inducing antibody or not) comprises a hydrophilic polymer such as one comprising polyethylene glycol.

Use of such hydrophilic polymer comprising photolabile groups assists in masking of the antibody where reduction in immunogenicity is desired. The use of such hydrophilic polymer comprising photolabile groups is particularly effective in the case of antibody fragments as an increase in half-life following administration can result. This is especially useful when the fragment is of a molecular weight which permits excretion via the kidneys as this does not occur in the fragments which have been reversibly inhibited by hydrophilic polymer containing photolabile group such that the molecular weight reduces or prevents renal excretion.

Hence in a favoured aspect, this invention provides a reversibly inhibited antibody or aptamer wherein reversibly inhibition is caused by covalently bonding thereto one or more usually more than one of photolabile groups which comprise a hydrophilic polymer which is preferably a polyethylene glycol.

The reversibly inhibited antibodies or aptamers may be administered systemically or locally. In either case a sterile aqueous system is favoured. Systemic administration may be intravenous administration. Local administration may be into the tumor (intratumoral) or into tissue near the tumor (peritumoral) including into minor vessels leading to the tumor.

The light cleavable moiety may be any comprising a light activatable group that absorbs photons and undergoes a chemical change resulting in release of the moiety from the antibody or aptamer. Presently favoured such moieties comprise a nitrophenyl group and preferably also comprise a polyethylene glycol residue. Favourably, the light is the near UV, i.e. UV light of about 320-380 nm.

Illumination at the desired site may take place once but more usually will occur on a number of occasions. The enhanced half-life of the reversibly inhibited antibodies generally avoids the need for continuous infusion and administration once or repeatedly at relatively lay intervals is contemplated. However, repeated illumination can lead to generation of reactivated antibodies, for example, illumination at intervals of 5 times a day to once a month can be appropriate. It is possible to employ larger doses than would be needed for the parent antibody, as toxicity issues are reduced. When the reversibly inhibited antibody is photoreactivated, its concentration can be highest in the locus of a tumour with lower concentrations and lower toxicity elsewhere. When low molecular weight fragments are regenerated, they can be excreted via the kidney, which also aids in reducing systematic toxicity.

The reversibly inhibited antibody may be prepared by treatment with a reactive derivative of the light activatable group, for example an acid halide derivative such as an acid chloride or an activated ester or the like.

Reversibly inhibited aptamers may be employed in analogous manner to antibody fragments. Hence they may be reversibly inhibited forms of aptamers which bind to cancer cells or cells of the immune system as described above in respect of antibodies.

Hence particularly apt reversibly inhibited aptamers are those which upon illumination regain the ability to bind to CD47, CD3, nucleolin, PD-1, PD-L-1 and the like. When an anti-CD47 antibody or aptamer is employed which leads to apoptosis of the cancer cell, the resulting apoptotic cells can lead to stimulation of the immune system to remove the cells by phagocytosis.

The light cleavable moiety or moieties employed with aptamers will aptly comprise a hydrophilic polymer as described above.

The invention also provides a reversibly inhibited anti-CD47 antibody or aptamer which antibody or aptamer causes phagocytosis and/or apoptosis of a cancer to which antibody or aptamer is bound one or more photolabile molecules which when subject to light are released from antibody or aptamer which thereby regains its ability to cause phagocytosis or apoptosis.

Suitable antibodies for reversible inhibition also includes those approved by the FDA for anti-cancer use or human trial at the filing date herein which lead to apoptosis followed by phagocytosis of cancer cells or by lysis followed by phagocytosis.

In particular, the present invention provides the use of the reversibly inhibited antibody hereof for the treatment of a cancer. Such cancers are described herein after, but in particular include those which present at or near the surface of the skin, for example skin cancers such as melanoma, or other cancers presenting at or near a bodily surface such as the skin or an internal bodily surface such as the oesophagus, stomach, rectum, colon, bladder, cervix or the like where illumination may be introduced without need to pierce the body. Ocular tumours such as ocular myeloma are similarly treatable.

The present invention also relates to analogous methods of treating diseases using reversibly inhibited antibodies or aptamers described in relation to the treatment of cancer.

If desired, the targeting function or a bio- or multi-function binding material may be a non-antibody or aptamer ligand of a receptor on the cell to be targeted.

### DETAILED DESCRIPTION OF THE INVENTION

In addition to the aspects set forth in the Summaries of the Invention set forth above, the following will assist the skilled person in respect of the present invention.

### Hydrophilic Reversibly Inhibited Antibodies and Fragments

The present invention provides antibodies or aptamers suitable for use in the treatment of a cancer, preferably a solid tumour, which comprises an antibody or aptamer thereof to which has reversibly bound one or more suitably more than one photolabile moieties which comprise a hydrophilic polymer such that the antibody or aptamer is inactive until photolabile moieties are released upon irradiation.

The antibodies (including fragments and aptamers) to be masked may be as described in the sections hereof in respect of stimulators of the Innate Immune System and of Stimulators of the Adaptive Immune System set out below.

The number of photolabile moieties employed may be any which results in rendering the antibody or aptamer inactive. Generally from 1 to 75 photolabile moieties are employed but the number depends on the size of the antibody or aptamer and the size of the hydrophilic polymer.

Whole antibodies normally employ a greater number of photolabile moieties than do fragments or aptamers. More photolabile moieties employing lower molecular weight polymers are generally required as compared to the number employed with higher molecular weight hydrophilic polymers.

When complete antibodies are to be reversibly inhibited often from 10 to 65 photolabile moieties are employed. Thus for example at least 20, at least 30, at least 40 or at least 50 such moieties are employed. Often not more than 60, not more than 50, not more than 40 or not more than 30, for example not more than 20 such moieties are employed.

In cases where the molecular weight of the hydrophilic polymer is lower, for example from 1 kDa to 20 kDa about 10 to 65 photolabile moieties are employed, for example 30 to 50 such moieties.

In cases where the molecular weight of the hydrophilic polymer is higher, for example 20 kDa to 60 kDa, a lower number of photolabile moieties is employed, for example 5 to 40 such as 10 to 30, for example 15 to 25.

A smaller number of higher molecular weight photocleavable moieties is sometimes more apt, as regeneration of the active antibody may occur more quickly.

When fragments or aptamers are employed a smaller number of photolabile moieties are employed depending on the molecular weight of the fragment or aptamer. The reduction in number from those set out above tends to be proportional to the reduction in molecular weight in comparison to an intact antibody.

In the case of antibodies and their fragments, the number of photolabile moieties reflects the number of lysine residues in the antibody or fragment as this amino acid is more readily derivated than, for example, serine or threonine or cysteine.

The number of photolabile moieties sometimes results from treating the antibody or fragment with an excess of reagent comprising the photolabile moiety so that accessible lysine groups are substituted. More extensive reaction can also lead to substitution of accessible serine and threonine groups or cysteine if desired.

When employing particularly small fragments such as scFv or diabodies the number of photolabile moieties may be small, for example 1 to 10, such as 2, 3, 4, 5, 6, 7 or 8 and may correspond to the number of lysine residues present in the fragment. In cases where lysine residues are remote from the binding site photolabile moieties may be bound threonine or serine residues present. Such smaller numbers are similarly contemplated for use with aptamers.

When employing such small fragments or aptamers the molecular weight of the hydrophilic polymer will normally be chosen to ensure that the molecular weight of the reversibly inhibited fragment is greater than 65 kD, more suitably greater than 70 kD or greater 80 kD.

Although any convenient biocompatible hydrophilic polymer may be employed it is presently preferred to employ a polyethylene glycol containing polymer. This polymer may also contain other components, for example polyoxypropylene as long as the polymer remains hydrophilic.

Presently it is preferred to employ a polymer which is a polyethylene glycol. This may be attached to the rest of photolabile moiety via a terminal oxygen of the polyethylene glycol or via an alternative such as a sulfur, amino, carboxy or other convenient group including spacing groups if desired. The terminus of the polyethylene glycol can be an OH group or more aptly an OH group capped by a convenient capping moiety such as methyl, acetyl or the like or the terminal OH can be replaced by a convenient moiety such as carboxyl acid, ester or the like. These linking and terminal groups may reflect the availability of suitable polyoxyethylene intermediates rather than an effect on the activity of the reversibly inhibited antibody or fragment as their effect is not believed to be dominant. Alternatively, if desired the polyethylene glycol can be terminated in a cytotoxic agent such as a commonly employed chemotherapeutic agent or by a T-cell stimulating small molecule (molecular weight less than 1 kDa).

The polyethylene glycol may be branched. The polyethylene glycol may be linear.

The molecular weight of the polyethylene glycol is chosen so that the reversibly inhibited antibody has a molecular weight that permits ease of formulation and distribution and penetration on administration.

It is believed that for such reversibly whole inhibited antibodies molecular weights of less than 1,000 kD, less than 900 kD, less than 800 kD, less than 700 kD, less than 600 kD, less than 500 kD, less than 450 kD, less than 400 kD, less than 350 kD, less than 300 kD or less than 250 kD are apt. It is believed that reversibly inhibited antibody fragments molecular weights of less than 500 kD, less than 400 kD, less than 350 kD less than 300 kD, less than 250 kD, less than 200 kD, less than 150 kD are less than 125 kD are apt. It is believed that for reversibly whole inhibited antibodies molecular weights of greater than 200 kD, greater than 250 kD, greater than 300 kD, greater than 350 kD, greater than 400 kD, greater than 450 kD, greater than 500 kD, greater than 600 kD or greater than 650 kD are apt. It is believed that reversibly inhibited antibody fragments of molecular weight greater than 70 kD, greater than 80 kD, greater than 90 kD, greater than 100 kD, greater than 110 kD, greater than 120 kD are apt.

The molecular weight of reversibly inhibited binding materials or the binding materials themselves may be determined by gel fliltration chromatography, ultracentrifugation, SDS PAGE electrophoresis etc.

The molecular weight of the polyethylene glycol is chosen to produce such suitable molecular weights in the reversibly inhibited antibody or aptamer.

It is believed that use of such by hydrophilic polymer can lead to a reduction in unwanted effects of circulating antibody or aptamer coupled with the desired activity at the locus of the cancer to be treated when the photolabile group are cleaved upon irradiation. Such antibodies or fragments or aptamers may be effectively masked when hydrophilic polymers become reversibly attached.

The reversibly inhibited antibody or fragment or aptamer may be any which prior to masking possessed anti-cancer effects especially in respect of a solid tumour.

Provision of a hydrophilic polymer coating can assist in reducing the immunogenicity of the thus masked antibody, fragment or aptamer.

The masked antibodies, their fragments or aptamers may be used as described in the section entitled “Methods of Use” and may be prepared as described in the section entitled “Preparative Methods”.

The use of covalently binding cleavable moieties as described herein can be applied with advantage to antibodies including fragments and aptamers which have demonstrated effectiveness in animals and in particular humans but which were found to possess systemic toxicity that reduced their suitability for clinical use and to fragments and aptamers which would benefit from an increase in serum half-life.

The antibodies are generally masked by covalently binding the photolabile moiety to amino acids in the antibody or fragment which have a substitutable group in their side chains, for example NH₂, OH, SH or CO₂H group, particularly NH₂ or OH and most preferably NH₂.

Aptamers may be marked by covalently binding the photolabile moiety to a hydroxyl group or a base or to a hydroxyl group already substituted by a readily derivatisable group such as an amino alkyl group, for example a 166 carbon amino alkyl group such as an amino ethyl group.

Most suitably covalent binding in antibodies or fragments occurs to lysine. Hence a favoured sub-set of antibodies or fragments to be reversibly inhibited are those having a plurality of lysine groups present. A preferred sub-set of such antibodies or fragments are those which possess lysine groups in the hypervariable region, for example within one or more of the CDR1 CDR2, CDR3, CDR4, CDR5 or CDR6 regions or within 10 amino acids of those regions.

In addition photolabile moieties may be covalently bound to hydroxyl or thiol residues present in the antibody or fragment but this is not presently contemplated as preferred.

When an antibody has a larger number of reactable sites the photolabile moiety suitably may comprise hydrophilic polymers (such as polyethylene glycols) in the lower part of the molecular weight range set out above as masking may be provided with a larger number of lower weight hydrophilic polymers (such as polyethylene glycols).

When an antibody, and in particular a fragment, has a lower number of reactable sites the photolabile moiety may suitably comprise hydrophilic polymers (such as polyethylene glycol) in the higher part of the molecular weights set out above as masking may be provided with a smaller number of lower molecular weight hydrophilic polymers.

### Stimulators of Innate Immune System

Antibody or aptamer stimulators of the innate immune system (such as an anti-CD47 antibody) for use together with an antibody or aptamer stimulator of the adaptive immune system such as a T-cell activating antibody (such as an anti-CD3 antibody) need not be reversibly inhibited if of low systemic toxicity.

However, many anti-CD47 antibodies and aptamers benefit from reversible inhibition as this can help avoid depletion by the bodily sink of CD47, can help reduce side effects and for antibody fragments and aptamers can improve half-life whether for use together with a stimulator of the adaptive immune system or not.

These benefits are aptly achieved by the use of one or more usually a plurality of photolabile moieties which comprise a hydrophilic polymer such as a polyethylene glycol.

When a plurality of amino acid residues such as lysine in an antibody or its active fragment are substituted by such photocleavable moieties the anti-CD47 antibody or its active fragment can be masked from bodily systems so that unwanted side effects are reduced and, particularly for fragments, circulatory half-life is extended. Similarly placement of a single photocleavable moiety at the binding site may be effective although not presently preferred.

Anti-CD47 antibodies can induce phagocytosis of cells directly by inhibiting the CD47-SIRPα binding between the cancer cell and phagocyte. This generally requires the presence of the Fc region with effector function on the antibody. Hence for use in the present invention where direct phagocytosis is desired the whole anti-CD47 antibody (as opposed to a fragment) is employed.

When used together with a reversibly inhibited anti-CD3 antibody or like T-cell activating antibody, the anti-CD47 antibody may be reversibly inhibited or not reversibly inhibited. In the latter case the anti-CD47 antibody will preferably be one which has been selected for reduced ability to cause side effects such as depletion of red blood cells or employed in a manner where such side effects are reduced, for example by a pre-treatment to deplete old blood cells, allowing younger blood cells to be introduced and then administering the therapeutic dose of antibody.

Directly phagocytic anti-CD47 antibodies which have been selected to result in reduced side effects include those described in references.

Directly phagocytic anti-CD47 antibodies which may be used in a manner where side effects are reduced include those described in references.

However it is presently believed that reversibly inhibited directly phagocytic anti-CD47 antibodies can be of benefit in the present invention. Suitable antibodies for reversible inhibition include those described in references.

The antibody for use may be a bispecific antibody if desired. Suitable antibodies include those described in references.

When the anti-CD47 antibody is selected to indirectly cause phagocytosis by first inducing apoptosis of the cancer cell, the whole antibody or a binding fragment may be employed (as Fc effector function is not mandatory).

The complete antibody may be reversibly inhibited if desired but it is believed that the use of reversibly inhibited fragments of agonist antibodies can be of particular use in causing apoptosis of cancer cells (which can then be phagocytosed).

As indicated above such fragments can have bound thereto a plurality of photolabile moieties which comprise a hydrophilic polymer which is preferably a polyethylene glycol.

The unmodified antibody fragments themselves benefit from better penetrability than the whole antibody but suffer from a shorter half-life. The reversibly inhibited fragments once photoactivated at the locus of a solid tumour can at least in part regain penetrability without the bulk of the administered reversibly inhibited antibody suffering from a short circulatory half-life.

The anti-CD47 fragment for reversible inhibition may be any type which possess apoptosis inducing properties such as a F(ab¹)₂, Fab, and in particular scFv, non-covalently linked scFv dimer (diabody), and covalently linked scFv (including s-s linked diabodies) and nanobodies.

Like whole antibodies, fragments for use herein can induce both phagocytosis and apoptosis if they also carry an agent which induces phagocytosis, for example by fusing a scFv to TNF related apoptosis inducing ligand as described by Wiersmaet al, Br. J. Haematology, 2, 304-307 (2013).

Suitable antibody fragments for use include those described in references ? to ? below.

In use the photoreactivatable antibody or fragment may be administered, for example by intravenous administration.

If a photoreactivatable anti-CD47 antibody or fragment is employed it may then be reactivated by exposure to light, particular near UV.

When used to enhance the effectiveness of a T-cell activating antibody this may be done by administration of both antibodies and subsequent illumination.

In analogous manner to use of directly phagocytic anti-CD47 antibodies, this invention also relates to analogous anti-CD10 and anti-CD19 antibodies which may be used together with the reversibly inhibited T-cell inducing antibody (such as anti-CD3 antibody). In such uses the anti-CD10 or anti-CD19 antibody may be advantageously reversibly inhibited in the manner described for anti-CD47 directly phagocytic antibodies.

Aptamers effective against CD47 may be reversibly inhibited and employed as described in respect of anti-CD47 antibody fragments. Such reversibly inhibited aptamers may be used in monotherapy but are also envisaged for use together with a stimulator of the adaptive immune system such as an anti-CD3 antibody or anti-PD-1 or PD-L-1 antibody.

Reversibly inhibited anti-nucleolin antibodies or aptamers may be employed owing to the wide occurrence of surface nucleolin on cancer cells. Enhancement of the serum half-life of antibody fragments and aptamers by reversible PEGylation as described herein is particularly advantageous and leads to apoptosis of the cancer cells.

When treating melanoma, co-targeting CD271 can assist in supressing metastasis. An antibody employed in treating cancer may be an anti-CCR4 antibody as aptamer. Such antibodies may be reversibly inhibited as described herein.

### Stimulators of the Adaptive Immune System

Antibody or aptamer stimulators of the adaptive immune system may be those which cause

T-cells to become more effective in destroying cancer cells. This may result from use of reversibly inhibited antibody which causes an expansion of the number of T-cells, by use of reversibly inhibited antibody which causes T-cells to become more effective in killing cancer cells, or by use of a reversibly inhibited antibody which inhibits pathways employed by cancer cells to evade T-cells.

As is generally the case throughout this document, in the above respect, the term antibody extends to fragments as well as complete antibodies and to bifunctional (and multifunctional) antibodies and to fragments.

One class of antibodies useful for T-cell expansion and/or increase in effectiveness are anti-CD3 antibodies.

One class of antibodies useful in inhibiting pathways which aid cancer cells in avoiding T-cells are anti-PD-1 and anti-PD-1L antibodies.

Antibodies which are preferred stimulators of T-cells for use in the treatment of cancer according to this invention are reversibly inhibited anti-CD3 antibodies.

The anti-CD3 antibody may be a complete antibody containing two Fab regions and Fc portions. In such cases either or both Fab portions may bind to CD3 expressed on a T-cell. In such cases the antibody may or may not be modified to enhance binding to a tumor cell. Also such antibodies may be modified to include additional Fab and/or Fc portions.

Well known antibodies which may be reversibly inhibited in accordance with this invention include OKT3 and UCHL-1. These antibodies may be modified to bind more effectively to a tumor cell by adding a function which recognises a receptor expressed by a tumor cell before being rendered reversibly inhibited.

For example the OKT3 and UCHL-1 antibody may be labelled with folate if the cancer to be treated expresses folate for example folate positive breast or ovarian cancers.

Similarly the OKT3 or UCHL-1 antibody may be labelled with a binding function against an epitope expressed on a cancer cell such as C19. In such cases the binding function can be an antibody or fragment thereof.

In all of the above instances the antibodies for use will be reversibly inhibited by covalently binding thereto light cleavable hydrophilic moieties as described herein.

In order to enhance specificity analogous antibodies or fragments thereof for use may be a reversibly inhibited bispecific antibody which comprises are anti-CD3 Fab portion and one Fab portion against an epitope on a cancer cell.

The antibody may comprise an Fc portion so that it is complete antibody.

The present invention is particularly applicable to bispecific antibodies where one functionality is directed to enhancing T-cell cytotoxicity and another functionality is directed against an epitope present on a tumour cell.

The second functionality may serve simply to attach the bispecific antibody to the tumour cell so that the T-cell enhancing function is made available to the T-cell so that it may become activated in the locus of the tumour cell.

The second functionality may serve additionally to induce its own cytotoxic effect, for example leading to phagocytosis either directly or via induced apoptosis, for example by binding to CD47.

Bi-specific T-cell engagers (BiTEs) are a class of bispecific antibodies intended for use as anti-cancer agents by directing the patient's T-cell cytotoxic activity against a cancer. BiTEs are fusion proteins of two single chain variable fragments (scFvs) of different antibodies on a single polypeptide generally of about 50-60 kD. One of the scFvs binds to a CD3 receptor on the T-cell and the other to a tumour cell via a tumour antigenic determinant. Such tumour antigenic determinants may be specific to the tumour or may be presented by the generality of tumours.

The low molecular weight of the BiTEs can be advantageous because it permits penetration into tumours but can also produce the disadvantage that it leads to a short serum half-life which can make it difficult to demonstrate effectiveness, for example after administration by intravenous or bolas administration.

Protecting the BiTEs from rapid elimination by reversibly inhibiting the BiTE with cleavable moieties permit reactivation of the BiTE at the site of the tumour.

Thus the present invention provides in a BiTE to which is covalently photoreversibly linked moiety.

In this aspect of the invention the photoreversible moiety aptly comprises a hydrophilic polymer which favourably is a polyethylene glycol.

The molecular weight of the polyethylene glycol (or other hydrophilic polymer) is suitably chosen so that the molecular weight of the thus masked BiTE is greater than 55 kDa, more suitably more than 60 kDa, such as more than 65 kDa, favourably more than 70 kDa for example more than 75 kDa or more than 90 kDa when the one or more commonly more than one photolabile moieties are linked to the BiTE.

The molecular weight of the polyethylene glycol (or other hydrophilic polymer) is suitably chosen so that the molecular weight of the thus masked BiTE is suitably not more than 500 kDa, more suitably not more than 400 kDa, such as not more than 330 kDa, for example not more than 300 kDa or not more than 250 kDa when the one or more commonly more than one photolabile moieties are linked to the BiTE.

Since BiTEs are of significantly lower molecular weight than the whole antibody, the number of photolabile groups required is less than when a whole antibody is being masked. Thus 1-15 photolabile moieties may be present, for example 2-12 photolabile moieties such as 3, 4, 5, 6, 7, 8, 9 or 10 photolabile moieties.

At least one scFv in such reversibly inhibited BiTEs will be one which engages and activates T-cells such as those directed against CD3 or PD-1. Hence are apt class of BiTEs comprise a scFv against CD3. Hence another apt class of BiTEs comprise a scFv against PD-1. If a polyfunctional approach the BiTE may comprise scFvs against both CD3 and PD-1.

At least one scFv in such masked BiTEs will be directed against a tumour antigen.

The tumour antigen may be one preferentially expressed or overexpressed on the generality of tumour cells (with little or no expression on non-tumour cells).

Alternatively the tumour antigen may be one specifically expressed by a particular tumour type (with little or no expression on non-tumour cells).

The second scFv can be against CD47. Such scFv can bind to the CD47 on cancer cells merely to aid in locating the T-cell activating scFv or it may in addition it may itself produce a cytotoxic effect. The use of a scFv against CD47 enables treatment of the generality of solid tumours by use of the masked BiTE.

Thus in a favoured aspect the present invention provides a BiTE which comprises a scFv against CD47 and a scFv that induces T-cell activation, for example a CD3 scFv or a PD-1 scFv. More favourably either or preferably both scFvs are reversibly inhibited and especially are reversibly inhibited by the presence of photolabile moieties comprising a hydrophilic polymer such as a polyethylene glycol. The unmasked induce phagocytosis either directly by inhibition of CD47-SIRPα binding or indirectly by inducing apoptosis followed by phagocytosis.

It is also known that phagocytosis of cancer cells can lead to presentation of their epitopes in a manner which enables recognition by T-cells.

It is known that anti-CD3 antibodies can be employed to induce destruction of cancer cells by activation of T-cells and that the likelihood of cytokine disease when using anti-CD3 antibodies can be reduced by reversibly deactivating them so that they can be reactivated at desired locus only.

WO2014179132/EP2992089 discloses use of an anti-CD47 antibody which inhibits CD47-SIRPα interaction for sufficient time to permit phagocytic antigen presenting cells (phAPC) incubated with an antigen to phagocytose the antigen and thereafter contact the antigen with T-cells.

Anti-PD-1 antibodies have proved successful in treating a variety of tumours. However such antibodies (for example Pembrolizumab) are also associated with adverse effects which are immune related as the body can be induced to attack itself on inhibition of PD-1 on erythrocytes. Masking PD-1 antibodies by the provision reversible inhibition using photolabile groups comprising hydrophilic polymers (in particular polyethylene glycol) can assist in reducing the immune related side effects since only erythrocytes in the locus of the tumour to be treated become of enhanced reactivity.

Thus the present invention provides an anti-PD-1 antibody reversibly inhibited by the covalent attachment of one or more photolabile moieties which comprise a hydrophilic polymer (in particular polyethylene glycol).

The reduction in immune related side effects can be enhanced by use of a PD-1 which also comprises a functionality which binds the antibody to the tumour cell. Thus one favoured sub-set of PD-1 antibodies for use are bi-specific antibodies wherein the second functionality binds the bispecific antibody to a tumour cell.

The second functionality may be as described in relation to anti-CD3 antibodies.

The use of fragments may be as described in relation to anti-CD3 antibodies.

The reversibly inhibited BiTE may suitably contain a functionality directed against nucleolin in an analogous manner to the use of an anti-CD47 functionality.

Antibodies against CCR4 may also be employed for reversible inhibition, for example, mogamulizumab.

### Anti-Cancer Antibody Fragments

The present invention provides photoreactivatable antibody fragments which comprise an antibody fragment of molecular weight less than 62 kDa to which is covalently bound one or more light cleavable moieties such that the photoreactivatable antibody has a molecular weight greater than 70 kDa and when exposed to light that cleaves one or more of the photocleavable moieties and regains anti-cancer activity.

The antibody fragment may be any anti-cancer effective fragment of an antibody such as a Fab, scFv, nanobody or the like and may be joined to a further antibody fragment as long as the molecular weight of the joined fragments does not exceed 62 kDa, for example as in a BiTE.

Anti-cancer antibody fragments with molecular weights less than 62 kDa tend to have short serum half-lives which reduces effectiveness and/or requires frequent administration or even administration by continuous infusion. Reversibly increasing the molecular weight of the fragments as described herein leads to an improvement in serum half-life. This can be achieved without leading to a reduction in penetration into the tumour of the reactivated fragment which can occur if molecular weight was permanently increased by irreversibly binding. Penetration of the active fragment throughout the tumour mass is highly desirable as it enhances the likelihood of successful therapy including killing of cancer stem cells.

In the aspect of the invention where the antibody fragment is bifunctional (e.g. a CD19/CD3 or CD9/PD1 or other bifunctional fragment) both or only one function will be reversibly inhibited (for example if only one function is reversibly inhibited it will preferably not be that which binds to the tumor cell (for example in the preceding examples CD19 will not be reversibly inhibited).

As noted above the molecular weight of the fragment to be reversibly inhibited will not exceed 62 kDa. Thus the following examples are included amongst those suitable for use in this aspect of the invention: Fab (˜50 kD; VH, CH1, VL, CL1), scFv (˜24-28 kDa; VH, VL), nanobodies (˜12-15 kDa; VHH or VH), VhHs (˜12-15 kDa), V-NAR (˜12-15 kDa) diabody (˜50 kDa bispecific, 2HV, 2VL), bis-scFv (bispecific ˜55 kDa; 2 VHs, 2 VLs), BiTE (˜50-55 kDa, bispecific, 2 scFv), tandem scFv (˜60 kDa, two scFvs), miniantibody (˜50 kDa,), scFv-CH-CL-scFv (˜50 kDa) and the like. The skilled person understands that these classes of antibody fragments are well known with many examples having been disclosed as effective anti-cancer agents.

The number of photolabile groups that may be bound to the antibody fragment will be sufficient to increase the molecular weight to above 65 kDa, preferably above 70 kDa, for example above 80 kDa, such as 85 kDa, 90 kDa, 95 kDa or 100 kDa.

If the antibody fragment is monofunctional it may be against any tumor cell surface marker which upon binding leads to killing of the cell, for example by leading to phagocytosis, apoptosis (which may lead to phagocytosis) or the like. Hence suitably the monofunctional antibody fragment may be an anti-CD47 fragment or an anti-CD19, anti-nucleolin. Similarly the fragment may stimulate the adaptive immune system leading to a cytotoxic effect on the tumor cell. Hence suitably the monofunctional antibody fragment may be an anti-CD3, anti-PD-1, anti-DP-1L or the like.

When the antibody fragment is bifunctional one function may serve to locate the fragment on a tumor cell. Such functions may be a 24-28 kDa, scFv fragment or a 12-15 kDa fragment such as a nanobody (camelid) or a VLHs or V-NAR (shark). These fragments may be directed to a tumor cell marker in conventional manner, for example a marker preferentially expressed on a tumor cell. Such markers include CD47, nucleolin, CD20 and other markers known to the skilled person.

In such bifunctional antibodies the second functionally may be directed to a second marker preferentially expressed on the tumor cell which may be as indicated above for the first marker of alternatively may stimulate the adaptive immune system, for example an anti-CD3 functionality. The anti-CD3 functionality is presently considered particularly favoured so that a preferred by functional antibody fragment is a BiTE (containing two linked scFv fragments) or an analogous bifunctional antibody in which functionality is a nanobody.

Particularly small fragments such as those of molecular weight 12-15 kDa such as a nanobody may have a smaller number of sites to which the photocleavable moieties may be easily attached. Such photoreactivatable fragments may thus contain for example 1, 2, 3, 4, 5, 6, 7, 8, 9 or 10 photoreactivatable moieties. In such cases if 1 moiety is attached it will aptly have a molecular weight of 60 kDa or more. If 2 moieties are attached they will have a total molecular weight of 60 kDa or more, for example 2 moieties each of molecular weight 30 kDa, or one of 20 kDa and one of 40 kDa and so on.

### Aptamer Aspects of the Invention

In a further aspect this invention provides a reversibly inhibited aptamer which comprises an aptamer to which is covalently bound one or more photolabile moieties comprising a hydrophilic polymer such as polyethyleneglycol such than on irradiation said moieties, are cleaved from the aptamer so that its cytotoxic effect on cancer cells is regained.

The light cleavable moieties can be as described in respect of antibodies and particularly as described in respect of antibody fragments. Generally aptamers benefit from increasing their molecular weight as described in respect of small antibody fragments so that in a favoured aspect the reversibly inhibited aptamers of this invention have a molecular weight not less than 60 kDa, preferably not less than 70 kDa, for example, not less than 75 kDa.

It is apt that the molecular weight of such reversibly inhibited aptamers is not greater than 200 kDa, favourably is not greater than 165 kDa, preferably is not greater than 150 kDa, for example, not greater than 120 kDa, such as not greater than 100 kDa.

The number of photolabile moieties bound to the aptamer and the molecular weight of the hydrophilic polymer such as polyethylene glycol will be employed to achieve the above molecular weights, for example as described in relation to low molecular weight antibody fragments herein.

The photolabile moieties can lead to loss of the specific binding properties of the aptamer by steric hindrance and/or by causing the aptamer to take on a non-folded or differently folded shape. Once the photolabile moiety or photolabile moieties are cleaved from the reversibly inhibited aptamer it is able to represent its binding face and regain activity, for example after refolding.

The skilled person is aware that large numbers of aptamers are available which have cytotoxic properties to cancer cells by binding to cancer markers on those cells. Because such aptamers may be synthesised by well-established chemical methods it is possible to modify a nucleoside if desired to already include the light cleavable moiety or to provide a point of ready attachment of such a moiety, for example by replacing a hydroxyl with an amino group or by substituting a hydroxyl group with an aminoethyl moiety or the like to present an easy target for attachment.

However it is presently envisaged that the aptamer need not be modified and that the photolabile moiety may be bound by chemical reaction, for example an acylation reaction.

This may take place in aqueous solution optionally containing minor amounts of a hydrophilic cosolvent such as DMF or DMSO. Such solutions may aptly be buffered, for example to pH 5-8.5, such as pH 6.5-8, and/or may optionally contain an acid acceptor such as a non-acylatable amine or potassium carbonate or the like.

The reversibly inhibited aptamer may be a reversibly inhibited RNA aptamer.

The reversibly inhibited aptamer may be a reversibly inhibited DNA aptamer.

The aptamer may be against any form of cancer but will be desirably against a cancer as referred to in respect of antibodies herein and in particular in respect of low molecular weight antibody fragments herein.

When monofunctional the reversibly inhibited aptamer may be one which upon illumination releases an aptamer which binds to a cancer cell and leads to its death or which binds to a cell of the adaptive immune system such as a T-cell and thereby stimulates the adaptive immune system to cause death of the cancer cell.

When bi-functional (or multifunctional) the reversibly inhibited aptamer may be one which upon illumination releases the bifunctional (or multifunctional) aptamer which binds to a cancer cell (with one or more of its functions) and leads to its death and/or which binds to a cell of the adaptive immune system such as a T-cell and thereby stimulates the adaptive immune system to cause death of the cancer cell.

Such aptamers may have functionalities directed to cell markers as described for antibody fragments and in particular in respect of BiTEs or of scFv fragments.

Hence particularly suitable aptamers for reversible inhibition include those which bind to CD47, CD3, PD-1, PD-L-1 and/or nucleolin.

Suitable aptamers include those described in Moreta et al, Cancers, 2018 (incorporated herein by reference) including anti-nucleolin aptamer AS1411 and anti-C4CL12 (SDF1) aptamer NOX-A12.

Reaction of an aptamer with a reactive derivative of a light cleavable moiety can lead to a mixture of products. In order to provide a more purified product, the product mixture can be treated with a source of its specific binding target and then unbound reversibly inhibited aptamer separated from the bound. The skilled person understands that column chromatography where the fixed phase has immobilised thereon the target marker may be employed. In various cases it may be useful to simply contact the aptamer product mixture with cells expressing the marker, allow any still non-inhibited aptamer to bind to the cells, separate the cells and obtain the purified reversibly inhibited aptamer. The cells may be of the cancer type to be treated or of non-cancerous cells which express the marker. Thus, for example, if the target marker is CD47 the cells employed may be red blood cells expressing CD47, or if nucleolin is the target marker epithelial cells expressing nucleolin may be employed.

Such purifications aid in ensuring minimisation of side effects from administering compositions of the reversibly inhibited aptamers.

Such compositions will be administered by injection including infusion, for example intravenously. Post administration the aptamer may be reactivated by photolytically as described in relation to antibody fragments.

Irreversible PEGylation of aptamers is known, thus for example, PEGylation of the *5-end has been employed to enhance serum half-life by resisting renal clearance and PEGylation of the *3-end has been employed to reduce nuclease degradation. However the known PEGylations have sought to retain the biological activity of the parent aptamer.

If desired the *5-end and/or the *3-end may be non-reversibly PEGylated and the aptamer also provided with one or more photocleavable moieties as described herein in order to render it inactive until illuminated as described herein so that side effects can be reduced.

If desired one or more of the hydrophilic polymer containing photocleavable moieties may also comprise a chemotherapeutic agent or T-cell stimulating agent as described herein.

Specific aptamers and preparatory methods are as described herein.

### Preparation of Reversibly Inhibited Antibodies or Fragments Thereof

The hydrophilic masked antibodies of the invention may be prepared by acylating the antibody with an acylating reagent of the formula

HP[A]-B—Oₙ—CO—X

wherein X is a leaving group to form an antibody substituted by one or more commonly a plurality of groups

HP-[A]-B—Oₙ—CO—

wherein HP is a hydrophilic polymer, n is 0 or 1, [A] is a photoactivatable group and B is a linker group such that an irradiation of the masked antibodies HP-[A]-B—CO₍ₙ₎-remnants are released from the antibody.

The number of HP-[A]-B—Oₙ—CO— groups present in the acylated antibody will be as indicated herein before.

The reaction will aptly occur under conditions suitable for acylation of proteins and especially antibodies, for example as disclosed in the Self et al references. This method may be modified by the skilled person, for example by restricting the amount of acylating reagent to provide the desired number of covalently bound photocleavable groups.

The leaving group X may be any suitable leaving group employed to enhance formation of an amide bond by a carboxylic acid. Typically X may be a chloride, bromide, mesylate, tosylate or the like.

We presently favour the use of the above acylating agents wherein Oₙ—CO—X is an O—CO—Cl group as they may be prepared by reaction of the corresponding OH compound by reaction with COCl₂, for example as disclosed in the Self et al references.

In the above acylating reagent, HP may be any polymer which aids in water solubility and has low antigenic potential so that the resulting masked antibody is water soluble and of sufficiently low antigenicity for use.

The polymer may be one containing —O—(CH₂—CH₂)—O— moieties or lactates and the like. Suitably the polymer comprises polyethylene oxide blocks and may favourably be a polyethylene glycol.

The polyethylene glycol may terminate in a hydroxyl group or the hydroxyl group may be substituted by any convenient capping group, for example by a C₁₋₆ alkyl group such as a methyl group, a C₁₋₆ acetoxy group such as a OCOCH₃ group or the hydroxyl group may be replaced by any convenient group such as a C₁₋₆ alkoxy carboyl group, a carboxylic acid group, an amino group or C₁₋₆ acylated amino group, SH group or C₁₋₆ acylated amino group of the like. The skilled worker will appreciate that the terminal group may be any that does not substantially alter the nature of the polyethylene oxide group and may suitably be chosen for ease of preparation of the acylating reagent. At present we prefer to employ PEGs terminating in an OH or OCH₃ group and in particular terminating in a OCH₃ group as the relevant intermediates are available and, in particular in respect of the OCH₃ terminated PEGs, and is synthesis of intermediates.

The HP may be linear or branched, for example linear or branched PEGs.

The molecular weight of the HP, for example the PEG, may be from 400 to 60,000 Da. Particularly PEGs are believed to have a molecular weight of at least 1 kDa, at least 4 kDa, at least 5 kDa, at least 10 kDa, at least 20 kDa, at least 30 kDa or at least 40 kDa. Particularly apt PEGs are believed to have a molecular weight of not more than 60 kDa, not more than 50 kDa, not more than 40 kDa, not more than 30 kDa or not more than 15 kDa (such weights being plus or minus 10%).

Suitable capped PEGs include those of molecular weight 2 kDa, 5 kDa, 10 kDa, 20 kDa, 30 kDa and 40 kDa (plus or minus 10%).

The PEGs may be straight chained or branched chain (including comb and star shaped).

The photoactivatable group [A] may be any which on irradiation, for example with UV light, cause chemical modification of the group B—O_{(H)} such that it covalent linkage to the antibody becomes broken.

The skilled chemist is aware that the literature is replete with photolabile moieties that may be employed to release protected groups.

It is presently preferred to employ aromatic groups [A] which are substituted by a nitro group ortho to the point of attachment of the group B. The skilled chemist is aware that ortho nitro-phenyl groups are well established in the art of photolabile release.

In the present case that HP group may be attached to such a nitrophenyl or like group in any convenient manner but at present it is preferred to attach it via a NHCO or OCO group, for example in a position para to the group B (and hence meta to the nitro group).

Such substituted nitrophenyl groups may be further optionally substituted by, for example, a second nitro group either to the group B, or by one or two or three (if no second nitro group present) F, Cl, B₆, O-alkyl_{(1-6c)}, O.CO,alkyl_{(1-6C)}, CF₃, CN or by a pendent or fused second phenyl group or the like.

At present we prefer to employ [A] as a phenyl group substituted ortho to the B group by a nitro group and para to the B group by a CO—NH—HP group or CO₂HP group.

The group B may be a —CH(R)—[CH(R)]-group where m is 0 or 1 and each R is independently hydrogen or alkyl or 1 to 4 carbon atoms. Most aptly B is a —CH₂—, —CH(CH₃)—, —CH₂CH₂— or —(CH₃)CH₂-group.

The reaction of the acylating reagent and the antibody or fragment thereof may occur in any convenient solvent in which the two reactants are soluble. An apt solvent in aqueous dioxane or the like. The reaction may take place at any convenient temperature and ambient temperature (for 18° C. to 28° C., more usually 20° C. to 24° C.) is apt. The reaction may occur in the presence of an acid receptor to take up the HX generated in the reaction.

The amount of the acylating agent will be determined by the number of sites desired to be acylated. In one convenient method the acylating reagent is present in excess so that all available sites for acylation are taken up. The number of available sites for acylation by a high molecular weight PEG containing light cleavable moiety may be lower than if a lower molecular weight PEG was employed.

It will be understood from the above that we favoured the acylating reagents of the above formula in which HP is a capped polyethylene glycol (e.g. a methyl capped polyethylene glycol) attached by a NH or O to a 4-CO-group of 2-nitro-phenyl ring wherein the 1 position is substituted by a CH(CH₃)CH₂O.CO.Cl group. The polyethylene glycol may aptly have a molecular weight of 1 kDa to 30 kDa, for example 10 kDa to 25 kDa, such as 20 kDa when a higher number of sites are to be acylated but may be of 10 kDa to 60 kDa, for example 15 kDa to 50 kDa, such as 20 kDa to 40 kDa is a lower number of sites is to be acylated, for example as may be the case in antibody fragments.

The bonding of the light cleavable moiety to the antibody is aptly an acylation reaction of a free NH₂, OH or SH group in the antibody. For antibodies most usually acylation of a free NH₂ group occurs, i.e. acylation of lysine residues is most commonly employed. For aptamers acylation most frequently occurs on such hydroxyl groups. Thus the acylation leads to linking of the light cleavable moiety to the antibody by formation of a new amide or ester linkage.

The reaction is generally performed under typical conditions for acylation of antibodies (or similar proteins) which are to retain the desired biological activity. Hence mild reaction conditions are employed.

Aqueous solvents are preferred which may also contain miscible solvents such as DMF or DMSO. The solution may be buffered or contain an acid acceptor such as K₂CO₃.

The acylation generally occurs in aqueous medium under non-extreme temperatures, for example at normal laboratory temperatures such as 15° C. to 30° C., more usually 18° C. to 25° C. although the skilled person may employ other temperatures that permit a sufficiently rapid reaction without unacceptable degradation.

It is normal to employ a base to remove acid generated by the reaction as is conventional in this type of chemistry. Favoured bases include amino compounds which as less readily acylated than the group to be acylated; thus for example tertiary amines may be employed when primary amino groups of the antibody are to be acylated and, less commonly, bases such as potassium bicarbonate may be employed for example if hydroxy groups or sulfhydryl groups are to be acylated.

An alternative reactive moiety for use in such reactive intermediate are carbonates such as O.CO.O.N═(COCH₂CH₂CO) (NHS-carbonate) which can be employed to derivatise an antibody or aptamer.

It can be convenient to employ commercially available reagents.

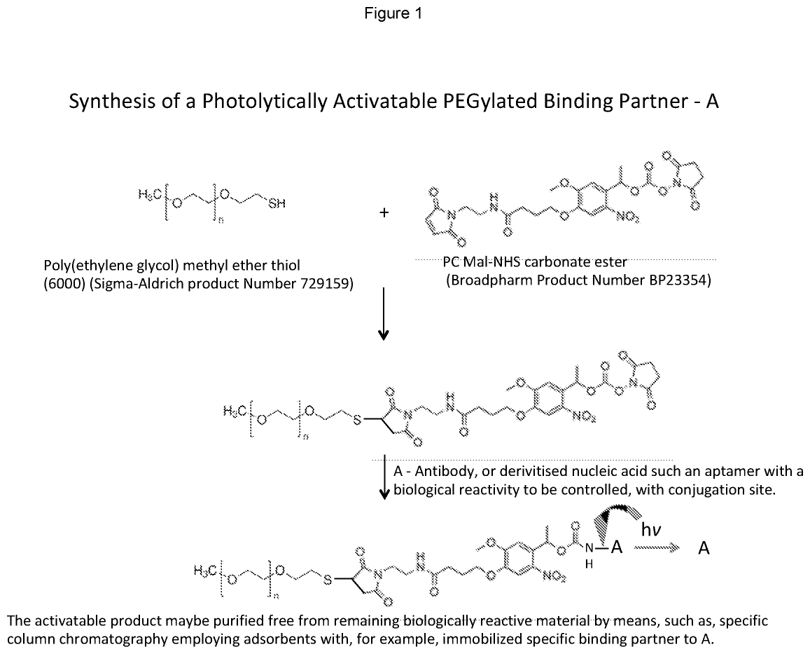

An example of a reaction scheme employing such a commercially available reagent is set out in FIG. 1. This illustrates how commercially available materials may be employed with convenience. The substitution pattern on the nitrophenyl ring can be modified as set out above, for example the methoxyl group can be omitted or replaced by another group moiety as chlorine or nitro or the like. Similarly, the substituent to the nitro group can be replaced by a polyethylene glycol containing substituent as described herein. However, for a skilled person wishing to do exploratory work, use of commercially available materials such as the PC Mal-NHS carbonate ester (Broadpharm Product Number BP 23354) or similar commercial materials avoids synthesis of intermediates. The Broadpharm website may be consulted for useful intermediates, as can that of Aldridge and other chemical suppliers.

If a high level of substitution is required the amount of acylating reagent may be in clear excess when based on the likely number of lysine or other acylatable residues in the antibody. If the sequence of the antibody is known then the skilled person is able to calculate the required weight of acylating reagent based upon the molecular weight of the reagent and antibody and employ an excess of, for example, 10% of acylating reagent based on the number of lysine residues.

If the sequence is not known the number of lysine residues may be estimated upon the number of amino acids in the antibody and the conventional relative abundance of lysine in such antibodies. To allow for variability in such factors in such cases an average of 30% or more may be employed.

When less than complete acylation of available groups is sought, the amount of acylation agent may be reduced accordingly.

When higher molecular weight hydrophilic polymers are present in the acylating reagent it is not always possible to acylate all lysine or other acylatable residues present in an antibody owing to steric or the like effects. Generally this results in a mixture of acylated products. Before use of the antibody, it can be advantageous to remove residual binding species (if any) by purifying the mixture of species by adscription abnormatography over immobilised target of the antibody. Thus for example an acylated anti-CD47 antibody can be purified on a column in which CD47 is presented, an acylated CD3 antibody can be purified on a column in which CD3 is presented, and so on.

This may be performed by column chromatography in a manner commonly employed by the skilled person to purify antibodies.

It will be understood that the number of lower molecular weight cleavable moieties that become attached to an antibody depends upon the molecular weight of that antibody. Thus an entire 1 Gg which has a molecular weight of about 150 ka will be able to accommodate roughly 50% more cleavable moieties than a F(ab¹)₂ fragment of molecular weight about 100 ka and roughly 3 times as many as a Fab fragment of molecular weight about 50 ka. Similarly a very low molecular weight fragment of for example 15 kDa will only have about very roughly 10% of the available sites for acylation than an antibody comprising two Fab regions and a Fc region.

Lysine residues often occur distributed over the whole antibody or its fragment at approximately 5-6%. Thus since the variable region comprises roughly 110-130 amino acids many antibodies contain 5-8 lysine residues in such the variable region. Hence antibodies have readily available acylatable residues on the binding site so that reversible inhibition is readily achieved by the method of this invention.

Acylation at sites other than the binding sites also play a role as this can lead to changes in presentation of the antibody reducing or eliminating binding.

The skilled person knowing or estimating the number of acylatable residues present in the antibody can choose the number of equivalents of the acylation reagent to employ appropriately. Ranging experiments with both the amount of reagent to employ and the weight of the hydrophilic polymer component may be performed in conventional manner.

Generally the higher the number of acylatable residues to be reacted the lower molecular weight of the polymer to be included in the cleavable moiety since very light molecular weights are not presently favoured.

In addition it is frequently impractical to put large numbers of high molecular weight groups onto an antibody owing to mutual steric hindrance. The skilled person will appreciate that although a full size antibody may have 50, 60 or even more lysine residues it is not practicable to attach a cleavable moiety comprising a hydrophilic polymer of molecular weight 20 kDa or more to that number of residues. However that number of acylations can be maximised if desired by using cleavable moieties where the hydrophilic polymer has a molecular weight of only 1 kDa.

Since the reagents and products generally sufficient solubility in an aqueous medium the skilled person is able to perform the acylations without needing to vary reaction conditions beyond the skilled person's conventional usage even though the acylation reagent can have different molecular weights. It can be sufficient to employ the same number of equivalents of the acylating reagent with relatively minor changes to concentrations, reaction times and temperatures and, if employed, purification columns.

For lower molecular weight fragments (for example of molecular weight not more than 62 kDa) fewer light cleavable moieties will be attachable since Fab, scFv, nanobodies, VhHs, V-NAR, diabodies, bis-scFv, BiTEs, tandem scFv, miniantibodies, scFv-CH-CL-scFv and the like have fewer acylatable lysine residues in proportion to their molecular weights.

The fragments may have 1 to 25 lysine residues but more usually 3 to 15 lysine residues suitable for acylation. However it is generally unnecessary to acylate substantially all such residues.

When reversibly inhibiting low molecular weight fragments such as those referred to above, the fragment is generally reacted with acylating agents wherein the hydrophilic polymer has a molecular weight of 4 kDa to 40 kDa, for example 5 kDa to 20 kDa such as 10 kDa. The quantity of reagent may be calculated to be sufficient to saturate the likely number of lysine residues and an average may be employed, for example up to 100%.

Where fewer than all available lysine residues are required for acylation the equivalents of acylating agent will be reduced to reflect this.

Even with small and very small fragments there is generally a plurality of lysine residues that may be acylated. Where the number is small the molecular weight of the polymer may be for example 10 kDa to 50 kDa, such as 20 kDa to 40 kDa and the equivalents of acylating agent chosen to provide a resultant molecular weight of over 70 kDa. In some instances it may be preferred to employ the lowest molecular weight polymer to achieve this.

The polymer present in the acylating agent may be straight or branched. Acylating agents containing branched polymers may be chosen when masking of the reversibly inhibited antibody from the immune system prior to activation is desired.

When a polyethylenegyclol is employed the remote end may be capped with any convenient molecular residue. Generally their residues will be small (molecular weight not more than 200 D) and aptly inert such as an alkyl, alkenyl, alkynyl, aryl or acyl group for example unsubstituted or substituted by residues which do not interfere with synthesis of the reversibly inhibited antibody or aptamer or the intermediates used in their production. More complex capping groups can be employed, for example anticytotic or immune cell stimulating agents are described herein.

When aptamers are prepared this often occurs in aqueous solution which may optionally contain solvent which a miscible but inert to the reaction conditions such as dimethylformamide, dimethylsuphoxide or the like.

The reaction conditions may be as indicated for antibodies but since acylation of the sugar hydroxyl groups on RNA aptamers or those inserted into DNA aptamers tends to be slower than acylation of primary amino groups, longer reaction times may be employed. If an amino group functionalised aptamer is to be acylated the reaction times are relatively shorter.

DNA aptamers may be prepared to have a hydroxyl group at a desired site (i.e. one or more nucleosides are “RNA” like as opposed to the convention “DNA nucleosides) is desired. This may provide a locus for attachment of the light cleavable moiety.

Both RNA and DNA aptamers may be synthesised to contain readily substitutable sites, for example on the sugar hydroxyl group in an RNA aptamer may be replaced by an amino or lower alkyl amino group, for example a OCH₂CH₂NH₂ group or the like, to provide a suitable locus for ready attachment of the light cleavable moiety.

In both RNA and DNA aptamers one or more bases may be modified to permit ready substitution by a light cleavable moiety.

Such modifications are well known to the skilled person and a number of contract aptamer manufacturers can supply such modified aptamers in normal commercial manner.

The use of modified aptamers in this way offers ready modification of a nucleoside within its normal binding part. This permits the use of a single light activated moiety if desired. In such a case the hydrophilic polymer such as the polyethylene glycol will have a molecular weight sufficient to render the reversibly inhibited aptamer a molecular weight greater than 72 kDa.

In similar manner two, three or more modifications may be made at or near the binding site.

It is also possible to substitute a hydrophilic polymer, for example to PEGylate, either reversibly or irreversibly, the 3′- and/or 5′-ends of the aptamer in addition to the binding site specifically reversibly attached light activatable moiety.

If one or two other such polymers, the weight of the three are such as to cause the weight of the thus substituted aptamer to exceed 70 kDa.

If only one hydrophilic polymer is attached it may have a molecular weight of, for example, 55 ka to 85 ka, for example 60 ka. If two hydrophilic polymers are attached they may have molecular weights which together add up to the previous figures, for example, one may have a molecular weight of 20 kDa and the other a molecular weight of 40 ka, or two may have a molecular weight of 30 kDa each. Similarly if three hydrophilic polymers they may each have a molecular weight of 20 kDa or two may have molecular weights of 10 kDa and one have a molecular weight of 40 kDa and so on.

When an aptamer has been modified to include an enhanced substitutable site, this may with advantage include a primary amino group which can be readily reacted in analogous manner to the amino group of a lysine in an antibody.

Both antibodies and aptamers may also comprise a non-light cleavable hydrophilic polymers substituents or substituents such as PEGs if desired as long as it does not prevent binding of the post-light cleavage antibody or aptamer. If it is desired to achieve high tumor penetration or rapid removal of antibody or aptamer from the body this option will not be favoured.

### Diseases of Microbial Origin

Many microbial pathogens such as bacteria, mycoplasma, viruses, fungi and protozoal parasites present markers on their surface which can bind ligands. This is in an analogous manner to how ligands bind to markers on cancer cells described herein.

The skilled person is able to identify relevant markers by known art methods, and the literature contains many examples of such markers. Antibodies may be raised against these markers in conventional manner and aptamers produced also in known manner and other binding partners for the chosen marker selected in known manner.

A marker of particular interest that occurs with frequency on microbial and viral pathogens is CD47. Hence antibodies against CD47 are herein contemplated for use in treating disease caused by microbial pathogens in analogous manner to that described for the treatment of cancer herein.

Microbial patterns of particular interest include the causative organisms for tuberculosis, candida, malaria, and lesmeiesis, as these have been difficult to treat but by virtue of expressing CD47.

Other microbial pathogens of interest include those with the potential to cause widespread infection, for example viruses such as influenza and polio. In the case of influenza, the target marker could be a neuraminidase or haemagglutinin epitope, but the invariant epitope at the stem junction would be also useful as a marker. The skilled person is aware of methods to prepare antibodies or aptamers against such epitopes.

To take advantage of enhanced T-cell response in treatment of the disease, the antibody or aptamer may also include a second function which enhances T-cell response to the microorganism. Thus, for example, the antibody may have one function which binds to a marker on the microorganism, for example an anti-CD47 function, and a second function which enhances T-cell response, for example an anti-CD3 function.

Suitable antibodies or aptamers with one or more, for example, 2 or 3 functionalities, are as described in relation to treating cancer.

Similarly, the antibody or aptamer may be rendered reversibly inhibited as described in respect to antibodies or aptamers for the treatment of cancer.

Since microbial diseases tend to be distributed throughout the body, local administration is less preferable to systemic administration so that administration intravenously is generally preferred. It has been shown that ant-CD3 treatment may result in systemic immunisation (Ellenhorn et al, Science (1988) 242, 569-571.

Similarly, since there may be no dominant locus for the disease, reversing inhibition of the antibody or aptamer is more effective by illuminating circulating blood. This may be done by use of a UV directed at a vein, for example in the wrist or by longer exposure to skin surfaces, for example the palm of the hand or sole of the foot. If thought desirable, an illuminated source, for example at the end of fibreoptics, can be inserted into a vein, for example, by use of an indwelling cannula (as can be done for treating cancers if desired).

The references hereafter also refer to various diseases, markers and antibodies for use in treating disease of microbial origin.

### Non-Antibody or Aptamer Binding Functions

The skilled person understands that there are many non-antibody or aptamer ligands which bind to markers on disease cells such as cancer cells.

This is seen, for example, in cancers such as breast cancer, which express markers to which folate binds. Hence when it is desired to target a cancer which expresses folate receptors, T-cell stimulatory antibody such as a CD3 antibody (or other referred to herein), it is possible to employ an anti-CD3 binding fragment (or indeed a whole antibody) and attach thereto folate to direct that binding fragment to the cancer cell. Aptamers may be employed in analogous manner when folated.

The increase in molecular weight of such folate substitute antibody fragment permits a longer residency time in the body.

Other non-antibody or aptamer binding functions include specific binding peptides to a marker on a disease cell such as a cancer cell. Hence specific binding peptides to CD47 may be employed to target T-cell enhancing functions such as an anti-CD3 antibody or fragment.

If it is wished to reversibly inhibit binding of the non-antibody or aptamer this may employ light-cleavable hydrophilic polymer as described herein.

Suitable antibodies for use with non-antibody or aptamer binding functions are as described in respect of antibodies or aptamers for treating cancer.

These antibodies or aptamers labelled with the binding materials may be used in the same way as corresponding bi-or multifunctional antibodies described herein.

Other diseases spread by mosquitoes are also amendable to treatment by this invention, for example dengue fever, Japanese encephalitis and the like.

The UV illumination may be from handheld or fixed lamps, for example employing a wavelength of 250-450 nm, more favourably 320-380 nm, for example 360-370 nm such as 365, 366, 367 or 368 nm.

Molecular weights may be determined in conventional manner, for example by gel filtration chromatograph. Suitable methods are generally available in the literature, for example, Ciaran O'Fogain et al in Chapter 2, pages 25-33, and Fee et al, Purification of PEGylated Proteins, in Methods of Biochemical Analysis series, Ed. J. C. Janson, Protein Purification: Principles, High Resolution Methods and Applications, Third Edition, 2011: chapter 14, doi 10.1002/9780470939932.

These are also incorporated herein by cross-reference. A suitable method is a column of Superdex G200 is poured and equilibrated according to the manufacturer's directions. Elution is stopped. An aqueous sample of the substance of which the molecular weight to be determined is carefully laid on top of the column. Elution is recommenced. Samples are taken from the bottom of the column and tested for the substance. The peak sample is recorded in terms of the elution volume required to cause it to flow through the column. The column is re-equilibrated and an aqueous mixture of marker proteins covering the range of interest is similar flowed through. A graph of molecular weight of the marker substances with their elution volumes is drawn from this data. The molecular weight of the substance to be determined is then read from graph by means of its elution volume.

Marker substances of the desired molecular weight may be included in gel filtration to assist in selecting fractions if desired, for example fluorescently labelled markers.

The skilled person understands that molecular weights of less than 60 kDa are found in the antibody fragments and their low molecular weight combinations herein.

Column chromatography may also be used to remove non-inhibited antibodies or aptamers, for example on a molecular weight basis or on an activity basis. Columns may contain solid phase, which presents the antigen/epitope to which the reversibly inhibited antibody binds on reactivation. Thus, for example, the column may contain immobilised CD3 or CD47 presenting materials or other marker materials. These may be proteins, for example, recombinantly produced CD3 or CD47 or other desired target protein of the antibody or aptamer, or may be a cell presenting the relevant marker. Hence, for example, erythrocytes may be used to remove still native anti-CD47 antibodies or aptamers, T-cells may be used to remove still active anti-CD3 antibodies and aptamers and so on.

In compositions for use, it is favoured to reduce the presence of still non-inhibited species as much as conveniently possible, for example the composition favourably will contain less than 10%, 9%, 8%, 7%, 6%, 5%, 4%, 3% or 2% of non-inhibited species (wt/wt) and preferably less than 1% (wt/wt) of non-inhibited species. Thus, it is preferred that the compositions are essentially free from non-reversibly inhibited species. Binding of the composition to target markers will thereby be reduced by at least 90%, 91%, 92%, 93%, 94%, 95%, 96%, 97% or 98%, favourably by at least 99% and preferably no residual binding of the composition to the target marker occurs.

### EXAMPLE 1

### Intermediate 1

Diphosgene (7.9 μL, 0.1316 mmol) was added to tert-butyl 4-(2-hydroxy-1-methylethyl)-3-nitrobenzoate (0.0185 g, 0.0658 mmol) in pyridine (5.2 μL, 0.06 μmol) and 1,4-dioxane (0.25 mL) and stirred at 25° C. for 15 minutes to provide the acid chloride in solution.

### EXAMPLE 2

### Reversibly Inhibited Anti-CD47 Antibody

An μg aliquot (100 μL) of the product of Example 1 was added to an anti-CD47 antibody (333 μL, 500 μg/mL, Invitrogen Lot 4347036) in NaHCO₃ (1 mL, 0.1 M) and then stirred for 18 hours at 25° C. The mixture was then dialysed against NaCl (0.9%) for 18 hours at 4° C. to give a colourless solution (2.5 mL). This contained anti-mouse antibody substituted by non-hydrophilic light cleavable moieties.

### EXAMPLE 3

### Intermediate 2

4-[2-[(2-Methoxyethoxy)methoxy]-1-methylethy]-3-nitrobenzoate (0.025 g, 0.084 mmol), benzotriazol-1-yl-oxytripyrrolidinophosphonium hexafluorophosphate (0.052 g, 0.100 mmol), N,N-disopropylethylamine (0.024 g, 0.184 mmol) and methoxypolyethylene glycol amine (0.439 g, 0.088 mmol, Mw 5000) in CH₂Cl₂ (5 mL) was stirred at 25° C. for 60 hours and then the solvent was removed in vacuo. The mixture was triturated with anhydrous Et₂O (10 mL) to form a pale cream solid which was centrifuged (3000 rpm, 60 seconds). Addition of Et₂O and centrifugation was repeated twice more to give a pale cream solid (0.375 g, 84%).

### EXAMPLE 4

### Intermediate 3

PEGylated 4-[2-[(2-methoxyethoxy)methoxy]-1-methylethyl]-3-nitrobenzoate (0.100 g, 0.0188 mmol), TFA (0.014 mL, 0.188 mmol) in CH₂Cl₂was stirred overnight at 25° C. and the solvent was removed in vacuo. The mixture was triturated with anhydrous Et₂O (10 mL) to form a colourless solid which was centrifuged (3000 rpm, 60 seconds). Addition of Et₂O and centrifugation was repeated twice more to give a colourless solid (0.063 g, 64%).

### EXAMPLE 5

Diphosgene (7.9 μL, 0.1316 mmol) was added to MEM deprotected PEGylated 4-[2-[(2-methoxyethoxy)methoxy]-1-methylethyl]-3-nitrobenzoate (0.027 g, 0.00526 mmol) in pyridine (5.2 μL, 0.06 μmol) and 1,4-dioxane (0.25 mL) and stirred at 25° C. for 15 minutes to prepare the acid chloride.

### EXAMPLE 6

The product of Example 5 was then added to anti-CD47 (333 μL, 500 μL/mL, Invitrogen Lot 4347036) in NaHCO₃ (1 mL, 0.1 M) and then stirred for 18 hours at 25° C. The mixture was then dialysed against NaCl (0.9%) for 18 hours at 4° C. to give a colourless solution (1 mL).

### EXAMPLE 7

### Demonstration of Reversing Inhibition

### Reactivation

One hundred and fifty microliters of the compounds of Examples 1 and 6 were put into separately labelled quartz cuvettes capped and exposed for 10 minutes as closely as possible to a Spectroline EN-16/F ultraviolet lamp (Spectronics Corporation, Westbury, N.Y.) with an emission peak of 365 nm. This type of lamp produces UV light over the wavelength range of 325-395 nm with a mercury vapour peak at 365 nm. The total UV-A irradiance of this hand-held lamp was 5.45 mWcm'2 at a working distance of 0.5 cm. These products were then labelled to indicate the contained photoreactivated Examples 1 and 6.

### Agglutination Test

Each of the wells of four rows of a 96 well microtiter plate received 50 μL phosphate buffered saline at pH 7.4. 1:2 serial dilutions are then made down each rows of: (a) Example 2 (b) illuminated Example 2, (c) Example 6, (d) illuminated Example 6 in each well by adding 50 μL of the antibody to be diluted into the first well of a row, mixing and then taking 50 μL of that mixture into the next well and so on. Once all the dilutions have been made, 50 μL of 1% gently re-suspended BALB/c blood cells (ENVIO S.B-999) was added to all wells. The contents of the wells were gently mixed. The plate was then covered and then left for the haemagglutination reaction to proceed at room temperature to allow sufficient recordable sedimentation to occur.

Following incubation pairs of wells receiving either illuminated or non-illuminated antibody conjugates were compared at each dilution down the rows of the plate the wells of the plate examined for haemagglutination in the classical terms of the inverse of red blood cell pellet formation, optimal pairs being scored for the appearance of RBC sedimentation.

| | | |
|---|---|---|
| (a) | Example 2 | 9 |
| (b) | Illuminated Example 2 | 1 |
| (c) | Example 6 | 9 |
| (d) | Illuminated Example 6 | 2 |

It was evident that both illuminated Examples 2 and 6 provided agglutination not seen by this testing system in their non-illuminated Examples 2 and 6. This demonstrated the anti-CD47 antibody had been effectively reversibly inhibited. This is demonstrative of results achievable with antagonistic anti-CD47 antibodies (for example such as those derived from B6H12 or its humanised versions).

### EXAMPLE 8

### Reversibly Inhibited Anti-CD47 F(ab¹)₂

The anti-CD47 antibody referred to in Example 2 was digested with papain in conventional manner to provide Fab fragments. The procedure of Example 6 was repeated but only using two thirds of the compound of Example 5 (34 μL aliquot and 40 μg Fab¹)₂ in 200 μL solution to yield a solution of reversibly inhibited Fab. It may be that this product had minor amounts of reversibly inhibited F(ab¹)₂ present.

### EXAMPLE 9

### Reversibly Inhibited CD47 Fab

A Fab fragment of the anti-CD47 referred to in Example 2 was prepared using a Pierce Fab kit. The method of Example 8 was repeated with this Fab (25 μg in 200 μL solution and a 20 μL aliquot of acid chloride).

It may be that the product contained minor amounts of the reversibly inhibited Fab fragment. This can be avoided by using ficin digestion in the presence of 4 nM cysteine in place of the papacin.

### EXAMPLE 10

### Reversibly Inhibited Anti-CD3 Antibody

The method of Example 6 is repeated using anti-CD3 antibody UCHT1 at OKT3 in place of anti-CD47 antibody to yield a reversibly inhibited anti-CD47 antibody.

### EXAMPLE 11

### Reversibly Inhibited Anti-CD3 Fab

A Fab fragment of UCTH1 which had been prepared from the UCHT1 anti-CD3 antibody by means of a Pierce Fab preparation kit (catalogue number 44905) was available. A solution (100 μL) of the Fab (45 μg in 300 ml) is treated with 100 μL of a solution of Example 5 in a manner described in Example 6.

### EXAMPLE 12

### Reversibly Inhibited Anti-CD47 Anti-CD3 Bispecific

A Fab fragment of the anti-CD47 antibody referred to in Example 2 and a Fab fragment of Example 11 of UCHT1 are prepared using a Pierce fragmentation kit.

The anti-CD47 Fab is reacted with the intermediate of Example 5 as described in Example 9. After dialysis and centrifugation of the solution of the reversibly inhibited Fab, in order to free from any residual Fab retaining CD47 binding affinity, the solutions are treated with washed BalbC erythrocytes in Alsever's solution (ENV105b-999).

Each of the two reversibly inhibited anti-CD47 Fab is coupled to UCHT1 Fab using SPDP in conventional manner. Each antibody is employed in solution estimated to contain 0.18 mg/ml and treated with a five-fold molar excess of SDPD.

### EXAMPLE 13

### Anti CD47—Anti CD3 Bispecific Antibody—Both Functions Reversibly Inhibited

An anti-CD47 Fab anti-CD3 Fab bispecific antibody is prepared from individual Fabs prepared as described in Examples 9 and 12 by treatment with SPDP in conventional manner.

The bispecific antibody is treated with the intermediate of Example 5 as described in Example 6 but reducing the amount of intermediate of Example 5 by half (50 μL aliquot). The solutions are purified as described in Example 12 to a solution containing the bispecific antibody in which both the functions were reversibly inhibited.

On the agglutination test these produce results as described above for Example 6 indicating that reversible inhibition of the CD47 function has occurred.
1. PCT Publication: WO 2009 024771 Materials and Methods for Treating Cancers which Express Folate Receptors, Self, Colin Henry; G B, Thompson, Stephen; G B, Publication date: 26 Feb. 2009
2. Self C H & Thompson S, Nature-Medicine, 1996, 2, 817, Light Activatable Antibodies: Models for Remotely Activatable Proteins.
3. Self, C. H. *Self A. C., Smith, J. A., Self, D. J., and Thompson, S.,*^{[a]} Light-Directed Activation of Human T-Cells Chem Med Chem 2007, 2, 1587-1590
4. Thompson, S.* Stewart, R., Smith J. A.,, Self C. H.,*^{[a]} Light Activation of Anti-CD3 in vivo Reduces the Growth of an Aggressive Ovarian Carcinoma, Chem Med Chem 2007, 2, 1591-1593
5. Thompson S., Fawcett M-C., Self C. H. “The construction of a functional photoactivatable cancer targeting bispecfic antibody conjugate” Chem Med Chem 2007, 2, 1-3
6. Thompson S., Dessi J., Self C H. “The construction and in vitro testing of photo-activatable cancer targeting folated anti-CD3 conjugates” in Biochemical and Biophysical Research Communications, 366 (2008) 526-531.
7. Thompson, S., Dessi, J., and Self, C. H., Preclinical evaluation of light-activatable, bispecific anti-human CD3 antibody conjugates as anti-ovarian cancer therapeutics, MAbs. 2009 July-August; 1(4): 348-356.
101. Kim et al, Tumor Biol.; 2008; 29:28-34
102. Gardai et al, Cell; 2005; 21:321-334
103. Jamieson et al, Blood; 2005; 106:3260
104. Kinchen et al, Current Biology; 18:R521-4
105. Mateo et al, Blood; 2002; 2882-2890
106. Manna et al; Cancer Research; 2004; 64:1026-1036
107. Creative Biolabs; humanized monoclonal antibodies to CD47, including anti-human CD47 therapeutic antibodies:—(C47Y1, TAB-273LC), (C47B91, TAB274LC), (C47B157, TAB275LC), (CD47B161, TAB276LC), (CD47B222, TAB277LC), (5A3, TAB278LC), (5A3, TAB278LC), (5A3-M3, TAB279LC), (TAB281LC, 282LC, 283LC, 284LC, 285LC, 286LC, 287LC, 288LC, 289LC, 290LC, 291LC, 292LC)
108. He Yam; Thesis, University of Groningen, 2016; on anti-CD47 fusion protein
109. US Patent Application 2017/0166645; on dual CD47/EGRF targeting
110. Creative Biolabs; Anti-Human CD47 Therapeutic Antibody scFu Fragments, TABs 291LC-S(P), 299LC-S(P), 302LC-S(P), 288LC-F(E), 284LC-S(P), 294LC-F(E), 286LC-S(P) and HPAP-0096-CN-F(E)
111. U.S. patent application Ser. No. 14/856,137, 2017/0071918
112. McCraken et al, American Association for Cancer Research; published online 2015, Jun. 29; Molecular Pathways, Activating T-Cells After Cancer Cell Phagocytosis From Blockage of CD47 “Don't Eat Me Signals”
113. WO 2013/119714 (non-agglutinating anti-CD47 antibodies)
114. WO 2016/05798
115. Golabovskaya et al; Cancers; 2017; 9:139 (doi 103390/Cancers 9100139)
116. Zong et al; Oncotarget; 2016, 7 (50), 83040-83050 (doi 18632/oncotarget 13349; PMID 27863402)
117. Zhiyuan et al; J. Immunol.; 2015; 195:661-671 (doi.org/10.4049/jimmuno1.1041719)
118. Kim et al; Leukemia; 2012; 26:2538-2545 (inhibition of myeloma cells)
119. Ngo et al; Cell Reports; 2016; 6:1701-1716 (inhibition of myeloma)
120. US 2013/0142786
121. Piccione et al; mAbs; 7:5946-5956; Bispecific CD47-CD20 Antibody
122. Gholamin; Science Translational Medicine; 2017; 9, issue 381, eaaf2968 (doi:

10.1126/scitranslmed.aaf2968)
123. Ingram et al; PNAS; 2017; 114:10184-10189 (doi 10.1073/pnas.17107776114)
124. Yuang et al; J. Thoracic Diseases; 2017; 9(2)E168-174
125. Weiskopf K; J. Clinical Investigation; 2016; 126(7); 2610-2620
126. Liu et al; Frontiers in Immunology, 2017; 8:1-17; (doi 10.3389/fmmu2017.00404)
127. Onol Res; 2017; 25(9); 1579-1587
128. BPS Biosciences; Anti-CD47 Antibodies and All Lines, 79118, 79302, 72514, 79105-1, 79105-2, 79065-1, 7095, 60689, 71292, 71249, 60602, 72059, 71169, 72044, 71177, 71127 and in Particular 79065-1 and 71177
129. Oncology Discovery; 2015 Nov. 4; Trillium Therapeutics on CD47
130. Trillium Therapeutics; Innate Immune Checkpoint, TTI-621, TTI-622
131. Surface Oncology; SRF231 Non-Agglutinating Anti-CD47 Antibody
132. Sockdosky et al; PNAS; 2016; 113:E2646-2654; Durable Responses to CD47 Blockade Require Adaptive Immune Responses
133. US Patent 2014/0363442
134. Yang et al; Cellular Physiology and Biology 2016; 40:126-136
135. Wiersma et al; University of Groningen; October 2014; Atlas of Genetics and Cytogenetics in Oncology and Haematology
136. Waclavicek et al; J. Immunol.; 1997; 159:5345-5354
137. Tsang et al; Proc. Natl. Aead. Sci.; 2013; 110:11103-11108
138. Pettersen; Apoptosis; 2000; 5:299-306
139. USP 2016/0257751
140. Dheilly et al; Molecular Therapy; 2017 25:523-533
141. Weiskopf; European J Cancer, 2017; 76:100-109
142. Kiss et al; J. Urology 2018; 159:e864 (doi 10.1016/j.juro.2018.02.2081)
143. Razaei et al; Nanomedicine; 2017; 12(6) (doi.10/2217/unm-2016-0302)
144. U.S. Pat. No. 9,221,908 B2
145. Kituchi et al; Leukemia Research; 2005; 4:445-450
146. EP2925782 A2 on CD47/CD19 bispecific antibodies
147. WO 2016/081423 A1 (non-hemagglutinating CD47 antibody)
148. Ngo et al; Cell Reports; 2016; 16:1701-1716
149. Willingham et al; Proc. Natl. Acad. Sci.; 2012; 109:6662-6667
150. Sockolosky et al; PNAS; 2016; 133:E2646-2654
200. Taki et al; PLOS One; December 2015; 10:e0144712 (PMC4682974, PMID26678395)
201. Huehis et al; Immunol Cell Biol; 2015; 93:29-296
202. Yamamoto et al; Biochem J.; 2012, July 1; 445(i): 135-144 (doi1042/BJ20120433)
203. Wittrup; Opinion; 2017; 3:615-620
204. Molgaard et al; Gene Therapy; 2017; 24:208-214
205. Rensch et al; Cancer Therapy Preclinical; December 2016; doi10.1158/1078-0432.ccl-16-0350
206. Kiprijanor et al; J Mol. Biol.; 1999; 293:41-56
207. Harwood et al; Oncoimmunology; 2108; 7(i); e1377874; doi 10.1080/2162402)(2017.1377874
208. Mazzoni et al; Cancer Res; 1996; 56:5443-9
209. Cheng et al; Int. J. Cancer, 2014; 136:476-486; doi10.1002/ijo.29007
210. Schmohl et al; Target Oncol.; 2016; 111:353-361
211. Baeuerie et al; Cancer Research; 2009; 69:4941-4944
212. Ross et al; PLOSone; 2017; 12(8): e0183390; doi10.1371/journal.pone.0183390
213. Yuraszeck et al; Clin. Pharm. & Therapeutics; 2017; 101:634-645
214. Ishiguro et al; Science Translational Med.; 2017; 9, eaa14291
215. Valasquez et al; Blood; 2017; 6, 741058; doi 10.1182/blood-2017-06-741058
216. Root et al; Antibodies; 2016; 5,6; doi 10.3390/antib5010006
217. Vey et al; Annals of Oncology; 2017; 28(5) V355-V371
218. White et al; Immunology, 2016; 77(13); Abstract No.4598
219. US 2017/0037130
220. WO 2017/134140
221. WO 2013/026837
222. WO 2014/114800
223. WO 2010/010413
224. WO 2016/016859
225. Durben et al; Mol. Ther.; 2015; 23:648-655
226. Wu et al; J. Hematol. Oncol.; 2015; 8:104; doi 10.1186/513045-015-0195-4
227. Leong et al; Blood; 2017; 129, 609-618
228. Hoseini et al; Blood Cancer J.; 2017; 7, e522
229. Sun et al; Science Translational Med.; 2015; 7(287):287ro70
230. Godpersen et al; Mol. Cancer Therapeutics; 2017 (May 12); doi 10.1158/1535-7163.MCT-16-0846
231. Li et al; Cancer Cell; 31:383-396
232. Hipp et al; Leukemia; 2017; 31:1743-1751
233. Horn et al; Oncotarget; 2017; 8, 57964-57980
234. Ferrari et al; J. Exp. Clin. Cancer Res.; 2015; 34:123
235. Borischwein et al; J. Immunother.; 2007; 30:798-807
236. Sternjak et al; Cancer Research; 2017; 13; doi 10.1158/1538-7445.AM2017-3630
237. Vaishanpayan et al; Prostate Cancer, 2015; 285193; doi 10.1155/2015/285193
238. Bacac et al; Clin. Cancer Res.; 2016; 22:3286-97
246. WO 2014/047231
247. Creative Biolabs BITES: CBL-BITES001-01, 001-11, 002-01, 002-11, 003-1, 003-2, 003-11, 003-12, 005-11, 008-02, 008-11, 008-12, and each of 009-01 to 009-63
248. Zhou et al; J. Cancer, 2017; 8:3689-3696
249. Patent Application CN 106084047
250. European Patent 1753783
251. Canadian Patent Application CA2879768
252. Osada et al; Cancer Immunol. Immunotherapy; 2015; 64:677-688
253. Huang et al; Clin. Immunol.; 2013; N9:156-168
254. Plitas et al; J. Clin. Oncol.; 2017; 34; doi 10.1200/JC0.2016.34.15.suppl.TPS3098
255 Kim et al, J Am Chem Soc. 2012 June 20; 134(24): 9918-9921, Synthesis of Bispecific Antibodies with Genetically Encoded Unnatural Amino Acids
301. Humphreys et al; Protein Engineering, Designs and Selection; 2007; 20:227-234
302. Mangla S; Ohio State University; 2016; Thesis on PEGylated antibody fragments
303. Doronin et al; Front. Immunol.; 15^{th} Conference Event Abstract; PEGylated Fab-fragments of GD2-Specific Antibody; doi 10.3389/conf.fimmu.2013.02.00193
304. Fee and Van Alstein; U. Waikato; doi 10.1.1.509.2865
305. Creative PEGworks and the Case for Protein PEGylation; Aug. 28, 2018 and Awwat et al; Engineering of Biomaterials for Drug Delivery Systems; doi 10.1016/B978-0-08-101750-0.00002-7
306. The website and/or catalogue of Broadpharm
401. Raymond et al; Proc. Natl. Acad. Sci.; 2018; 115:168-173
402. Bellmunt et al; Cancer Treatment Reviews; 2017; 54:58-67
403. Cancer Research UK; 6.2.2014; science blog; New Immunotherapy Drugs Hit the

Headlines
404. Hamanishi et al; Int. J. Clin. Oncol.; 2016; 21:462-473
405. ESMO 2010 Congress; 1.3.2017; on Anti-PD-1/PDL-1 Immunology in Lung Cancer
406. Alsaab et al; Fron. Pharmacol.; 2017; 8:561; doi 10.3389/fphar.2017.00561
407. Balar et al; Cancer Immunol. Immunother; 2017; 66:551-564
408. Feng et al; Cancer Letters; 2017; 407:57-65; doi 10.1016/j.canlet.2017.08.006
409. National Cancer Institute Staff; www.cancer.gov/news-eventscancer-currents-blog/2017/approvals-fda-checkpoint-bladder
410. Hwang et al; JAMA Oncol.; 2018; 4:253-255
411. Bilgin et al; Current Med. Res. Opinion; 2017; 33:749-759
412. U.S. Pat. No. 8,008,449 B2
413. Chang et al; AACR Therapeutics; 9.19.2017; doi 10.1158/0008-5472.CAN-16-3431
414. U.S. Pat. No. 9,567,399 B1
415. Maute et al; on high affinity PD-1 variants; Nov. 10, 2015; PNAS; doi 10.1073/pnas.1519623112
416. Bannas et al; Nanobodies Fron. Immunol.; 11.22.2017; doi 10.3389/fimmu.2017.01603
417. Stark et al; J. Biol. Chem.; 2009; 284:25612-25619
418. ABCORE; Single Domain Antibodies (Nanobodies) Supplier
419. Nekrasova et al; Nanobodies; La Cava Lab; 2017 poster
420. WO 2016/124781 A1
421. Palmeri et al; Proc. Nat. Acad. Sci.; 2015; 112:9418-9423
422. US 2016/0215050 A1
423. Creative Biolabs for antibody fragments
424. Mishra et al; Asian J. Pharm. SCI.; 2016; 11:337-348
425. Ablynx supplier of nanobodies
426. Pitcovski et al; Critical Reviews in Oncology/Hematology; 2017; 115:36-49
427. U.S. Pat. No. 8,563,269 B2
428. US 2016/0311903 A1
429. US 2014/0023664 A1
430. US 2012/0149061 A1
431. WO 2011/104565 A1
501. Haruta et al; Nucleic Acid Ther.; 2017; 27:36-44
502. Zhou et al; Oncotarget; 2016; 7:13446-13463
503. Boltz et al; J. Biol. Chemistry; 2017; 286:21896-21905
504. The Aptomer Handbook edited Klussman, Wiley
505. Mikat et al; RNA; 2007; 13:2341-2347
506. Morita et al; Cancers; 2018; 10:80; doi 10.3390/cancers10030080
507. Volk et al; Biomedicines; 2017; 5:41
508. Morita et al; Mol. Ther. Nucleic Acids; 2016; 5:e399
509. Fernandez-Garcia et al; Synlent; 2016-10-11
510. Velema et al; J. An. Chem. Sul.; 2018; 140:3491-3495
511. Trilink Biotechnologies website
512. Rafiq et al; Nature Biotechnology; 2018; 36:847; doi 10.1038/nbt.4195
513. Introduction to Practice Biochemistry; Gyorgy Hegyi et al; 2013; Eotvos Lorand University; Chapter 6
514. Winzore D J; Chapter 9; Physical Principles and Techniques of Protein Chemistry; Part 1, in particular 1A (edited by Sydney Leach)
515. Introduction to Analytical Ultracentrifugation; Beckman; Gred Ralston; U. Sydney
516. Chen et al; Virol J.; 2017; 14:189

1. A reversibly inhibited binding molecule which binding molecule enhances the effectiveness of the immune system to attack a disease such as cancer, to which binding molecule is bound one or more photolabile moieties comprising a hydrophilic polymer which when subject to light are released from the binding molecule which thereby regains its ability activate the immune system.

2. A reversibly inhibited binding molecule as claimed in claim 1 wherein the binding molecule is an antibody.

3. A reversibly inhibited binding material as claimed in claim 1 wherein the binding molecule is an antibody fragment.

4. A reversibly inhibited binding molecule as claimed in claim 1 wherein the binding molecule is an aptamer.

5. A reversibly inhibited binding molecule as claimed in any of claims 1 to 4 wherein the binding molecule activates the adaptive immune system.

6. A reversibly inhibited binding molecule as claimed in any of claims 1 to 5 which activates the innate immune system.

7. A reversibly inhibited binding molecule as claimed in claim 6 which causes phagocytosis of a disease-causative cell and in particular a cancer cell by inhibiting a don't-eat-me signal on the cell.

8. A reversibly inhibited binding molecule as claimed in any of claim 7 which is an anti-CD47 antibody.

9. A reversibly inhibited binding molecule as claimed in claim 5 which binds to CD3 or PD-1.

10. A reversibly inhibited binding molecule as claimed in any of claims 1 to 9 which has a molecular weight of less than 60 kDa.

11. A reversibly inhibited binding molecule as claimed in any of claims 1 to 10 wherein the light cleavable moiety comprises polyethyleneglycol.

12. A reversibly inhibited antibody or aptamer as claimed in claim 1 which antibody causes stimulation of the immune system to attack a cancer to which is bound more than one photolabile moieties comprising a polyethyleneglycol which when subject to light regains its ability to stimulate the immune system.

13. A reversibly inhibited antibody or aptamer as claimed in claim 12 which is an anti-CD47, anti-CD3, anti-PD-1, or anti-nucleolin antibody or anti-CD47, anti-CD3, anti-PD-1 or anti-nucleolin aptamer.

14. A reversibly inhibited antibody as claimed in any of claims 1 to 13 wherein the antibody is a fragment of molecular weight not greater than 62 kDa and the reversibly inhibited antibody fragment has a molecular weight greater than 70 kDa.

15. A reversibly inhibited aptamer as claimed in any of claims 1 to 13 which has a molecular weight greater than 70 kDa.

16. A reversibly inhibited binding molecule as claimed in any of claims 1 to 15 wherein the photolabile moiety is one which is cleaved from the binding molecule on exposure to UV light.

17. A reversibly inhibited binding molecule as claimed in claim 16 wherein the photolabile moiety is a nitrophenyl derivative.

18. A reversibly inhibited binding molecule as claimed in claim 17 wherein the nitrophenyl derivative is substituted by a polyethyleneglycol.

19. A reversibly inhibited binding molecule as claimed in claim 18 wherein the nitrophenyl derivative is substituted by a carboxylic acid ester or amide comprising polyethyleneglycol.

20. A reversibly inhibited binding molecule as claimed in claim 1 wherein the binding molecule is a bispecific antibody fragment of molecular weight less than 60 kDa and the reversibly inhibited BiTE has reversibly bound thereto photocleavable moieties comprising polyoxyethyleneglycol polymer such that the molecular weight of reversibly inhibited BiTE is from 70 kDa to 200 kDa.

21. A reversibly inhibited BiTE as claimed in claim 20 wherein the specificities are to CD3 and to CD47 or PD-1.

22. A reversibly inhibited binding molecule as claimed in claim 1 wherein the binding molecule is an antibody or aptamer described in a reference set out hereinbefore.

23. A method of treating malignant melanoma which comprises administering to a subject in need thereof an effective amount of a reversibly inhibited antibody according to any of claims 1-22 and illuminating a malignant melanoma tumour on the skin of the subject, whereby inhibition of the antibody is reversed.

24. A method of treating a malignant melanoma metastasis not on the skin which comprises administering to a subject in need thereof an effective amount of a reversibly inhibited antibody according to any of claims 1-22 and illuminating a malignant melanoma tumour on the skin of the subject whereby inhibition of the antibody is reversed.

25. A method of treating a first tumour which comprises administering to a subject in need thereof an effective amount of a reversibly inhibited antibody according to any of claims 1-22 and illuminating a second tumour in the subject whereby inhibition of the antibody is reversed.

26. A method of treating a cancer in a subject in need thereof which comprises administering an effective amount of a reversibly inhibited antibody according to any of claims 1-22 and illuminating a tumour of that cancer in the subject whereby inhibition of the antibody is reversed.

27. A method of treating a cancer in a subject in need thereof which comprises administering an effective amount of a reversibly inhibited antibody according to any of claims 1-22 and illuminating circulating blood of the patient whereby inhibition of the antibody is reversed.

28. An antibody of any of claims 1 to 22 for use in treating cancer.

29. An antibody of any of claims 1 to 22 for use in treating cancer by a method of any of claims 23-28.

30. A reversibly inhibited anti-CD47 antibody for use in the treatment of a solid tumour wherein the reversibly inhibited anti-CD47 antibody has covalently bound thereto photocleavable moieties which on illumination are cleaved to reverse the inhibition of the anti-CD47 antibody.

31. A reversibly inhibited anti-CD47 antibody according to any of claims 1 to 22 for use in the treatment of a solid tumour as set forth in any of claims

32. A reversibly inhibited anti-CD47 antibody for use according to either of claim 30 or 31 wherein the anti-CD47 antibody directly causes phagocytosis of the cells of the solid tumour.

33. A reversibly inhibited anti-CD47 antibody for use according to either of claim 30 or 31 wherein the anti-CD47 antibody causes apoptosis of the cells of the solid tumour.

34. A method of treating a cancer selected from a skin (bladder, cervical cancer, eye cancer, cancer of the buccal cavity, oesophagus, stomach, rectum, colon, brain, prostate and in particular malignant melanoma) in a subject in need thereof which comprises administering to said subject an effective amount of an antibody comprising an anti-CD47 binding function and an anti-CD3 binding function, either or both of which functions are reversibly inhibited by one or more covalently bound light-cleavable moieties which upon illumination are cleaved to reverse the inhibition and illuminating the cancer presenting at or near a bodily surface, and in particular a malignant melanoma tumour on the skin of the patient.

35. A method of treating a cancer according to claim 34 wherein both the anti-CD47 and anti-CD3 functions are reversibly inhibited.

36. A method of treating a cancer according to claim 34 wherein the anti-CD47 binding function causes apoptosis of the malignant melanoma cells when inhibition is reversed.

37. A method of treating a cancer according to claim 34 wherein the anti-CD47 binding function directly causes phagocytosis of the malignant melanoma cells when inhibition is reversed.

38. A method of treating malignant melanoma according to any of claims 34-36 wherein the reversibly inhibited antibody has a molecular weight of greater than 72 kDa and the antibody without the covalently bound light cleavable moieties has a molecular weight of less than 60 kDa.

39. A method of treating a malignant melanoma according to claim 37 wherein the reversibly inhibited antibody is a BiTe.

40. A method of treating malignant melanoma according to any of claims 34-38 wherein the anti-CD47 function is a nanobody (a camillid heavy chain antibody of molecular weight of less than 20 kDa, generally about 15 kDa).

41. A method of treating malignant melanoma according to any of claims 23-37 wherein an antibody or aptamer to PD-1 or PD-L-1 is also administered to the subject in need of treatment.

42. A method of treating an infection caused by a microorganism selected from bacteria, mycoplasm, virus, fungi or protozoal parasite by administering to a patient in need thereof an effective amount of an antibody as described in any of claims 34-41 and thereafter, illuminating the antibody to reverse the inhibition.

43. A method of treating an infection according to claim 42 selected from malaria, leishmeisis and candida wherein the antibody is as described in any of claims 35 to 41.
